(19) United States
(12) Patent Application Publication
(10) Pub. No.: US 2022/0202371 A1
(43) Pub. Date: Jun. 30, 2022

(54) BIOLOGICAL-DATA PROCESSING APPARATUS, BIOLOGICAL-DATA MEASUREMENT SYSTEM, AND RECORDING MEDIUM

(71) Applicants: Fumikazu HOSHI, Ishikawa (JP); Yuki Miyano, Tokyo (JP); Hideaki Yamagata, Kanagawa (JP); Shigenori Kawabata, Tokyo (JP); Nobuyori Nagaoka, Tokyo (JP); Taishi Watanabe, Tokyo (JP)

(72) Inventors: Fumikazu HOSHI, Ishikawa (JP); Yuki Miyano, Tokyo (JP); Hideaki Yamagata, Kanagawa (JP); Shigenori Kawabata, Tokyo (JP); Nobuyori Nagaoka, Tokyo (JP); Taishi Watanabe, Tokyo (JP)

(73) Assignees: Ricoh Company, Ltd., Tokyo (JP); National University Corporation Tokyo Medical and Dental University, Tokyo (JP)

(21) Appl. No.: 17/645,555

(22) Filed: Dec. 22, 2021

(30) Foreign Application Priority Data
Dec. 24, 2020 (JP) 2020-214933
Oct. 15, 2021 (JP) 2021-169911
Nov. 26, 2021 (JP) 2021-192184

(51) Int. Cl. A61B 5/00; A61B 5/248

(52) U.S. Cl. CPC A61B 5/7221; A61B 5/248; A61B 5/6802

(57) ABSTRACT

A biological-data processing apparatus includes a processor; and a memory that includes instructions, which when executed, cause the processor to execute performing an addition-averaging process every time an addition count of biological data reaches a predetermined count, the biological data being measured in response to a trigger signal associated with a stimulus applied to one or more parts; storing, in a storage, addition-average data resulting from the addition-averaging process performed for each of the stimulated one or more parts, in association with the addition count in the addition-average data; and performing a biological data process based on the biological data, by using the addition-average data corresponding to each of the stimulated one or more parts, the addition-average data being acquired by referring to the storage based on the addition count that is specified.

### CROSS-REFERENCE TO RELATED APPLICATION

The present application is based on and claims priority under 35 U.S.C. § 119 to Japanese Patent Application No. 2020-214933, filed on Dec. 24, 2020, Japanese Patent Application No. 2021-169911, filed on Oct. 15, 2021, and Japanese Patent Application No. 2021-192184, filed on Nov. 26, 2021, the contents of which are incorporated herein by reference in their entirety.

### BACKGROUND OF THE INVENTION

### 1. Field of the Invention

The present invention relates to a biological-data processing apparatus, a biological-data measurement system, and a recording medium.

### 2. Description of the Related Art

Conventionally, a biological-data measurement system is known for measuring biological data such as biomagneticfield data generated in response to a stimulus such as an electrical stimulus. In such a biological-data measurement system, in order to reduce noise included in weak biological data, there are cases in which an addition-averaging process is performed on biological data measured in response to trigger signals that are periodically generated.

Further, in order to improve the convenience in an electrocardiogram test involving an addition-averaging process, a configuration is disclosed in which an addition-averaging process is performed such that an electrocardiogram signal that matches a predetermined template is added, and an electrocardiogram signal that does not match a predetermined template is excluded from the addition (see, for example, Patent Document 1).
Patent Document 1: Japanese Patent No. 6555830

### SUMMARY OF THE INVENTION

According to one aspect of the present invention, there is provided a biological-data processing apparatus including a processor; and a memory that includes instructions, which when executed, cause the processor to execute performing an addition-averaging process every time an addition count of biological data reaches a predetermined count, the biological data being measured in response to a trigger signal associated with a stimulus applied to one or more parts; storing, in a storage, addition-average data resulting from the addition-averaging process performed for each of the stimulated one or more parts, in association with the addition count in the addition-average data; and performing a biological data process based on the biological data, by using the addition-average data corresponding to each of the stimulated one or more parts, the addition-average data being acquired by referring to the storage based on the addition count that is specified.

### BRIEF DESCRIPTION OF THE DRAWINGS

FIG. 1 is a diagram illustrating an example of the overall configuration of a biological-data measurement system according to an embodiment of the present invention;

FIG. 2 is a diagram for explaining an example of a configuration of a measurement apparatus according to an embodiment of the present invention;

FIG. 3 is a block diagram of an example of a hardware configuration of a computer according to an embodiment of the present invention;

FIG. 4 is a block diagram illustrating an example of a functional configuration of a measurement WS according to a first embodiment of the present invention;

FIG. 5 is a block diagram illustrating an example of a functional configuration of a data storage server according to the first embodiment of the present invention;

FIG. 6 is a block diagram illustrating an example of a functional configuration of an analysis WS according to the first embodiment of the present invention;

FIG. 7 is a flowchart illustrating an example of an operation of a measurement WS according to the first embodiment of the present invention;

FIG. 8 is a flowchart of an example of an operation of an analysis WS according to the first embodiment of the present invention;

FIG. 9 is a diagram illustrating an example of a screen for specifying a predetermined count and a total addition count according to the first embodiment of the present invention;

FIG. 10 is a diagram illustrating an example of a measurement screen and an operation screen according to the first embodiment of the present invention;

FIG. 11 is a diagram illustrating an example of an addition-average data list according to the first embodiment of the present invention;

FIG. 12 is a diagram illustrating an example of a display screen during estimation of an action current according to the first embodiment of the present invention;

FIG. 13 is a diagram illustrating an example of a display screen of an inappropriate result of estimating an action current according to the first embodiment of the present invention;

FIG. 14 is a diagram illustrating an example of a display screen of an appropriate result of estimating an action current according to the first embodiment of the present invention;

FIG. 15 is a block diagram of an example of a functional configuration of an analysis WS according to a second embodiment of the present invention;

FIG. 16 is a block diagram illustrating an example of a functional configuration of a measurement WS according to a third embodiment of the present invention;

FIG. 17 is a diagram illustrating a first example of a screen for specifying a predetermined count and a total addition count according to the third embodiment of the present invention;

FIG. 18 is a diagram illustrating a second example of a screen for specifying a predetermined count and a total addition count according to the third embodiment of the present invention;

FIG. 19 is a diagram illustrating an example of the overall configuration of the biological-data measurement system according to fourth to sixth embodiments of the present invention;

FIG. 20 is a block diagram illustrating an example of a functional configuration of a measurement WS according to the fourth embodiment of the present invention;

FIG. 21 is a block diagram illustrating an example of a functional configuration of a data storage server according to the fourth embodiment of the present invention;

FIG. 22 is a block diagram of a functional configuration diagram of an analysis WS according to the fourth embodiment of the present invention;

FIG. 23 is a flowchart illustrating an example of an operation of a biological-data processing apparatus according to the fourth embodiment of the present invention;

FIG. 24 is a diagram of a first example of a screen for specifying a predetermined count, a segment width, and a total addition count according to the fourth embodiment of the present invention;

FIG. 25 is a diagram of a second example of a screen for specifying a predetermined count, a segment width, and a total addition count according to the fourth embodiment of the present invention;

FIG. 26 is a diagram of an example of waveform data displayed on a measurement screen or an analysis screen according to the fourth embodiment of the present invention;

FIGS. 27A to 27C are diagrams of an example of the frequency spectrum displayed on the measurement screen or the analysis screen according to the fourth embodiment of the present invention;

FIG. 28 is a diagram illustrating an example of a segment addition data list according to the fourth embodiment of the present invention;

FIG. 29 is a block diagram illustrating an example of a functional configuration of a measurement WS according to a fifth embodiment of the present invention;

FIG. 30 is a block diagram illustrating an example of a functional configuration of a data storage server according to the fifth embodiment of the present invention;

FIG. 31 is a block diagram of a functional configuration diagram of an analysis WS according to the fifth embodiment of the present invention;

FIG. 32 is a flowchart of an example of an operation of a biological-data processing apparatus according to the fifth embodiment of the present invention;

FIG. 33 is a diagram illustrating a screen for specifying a predetermined count, a segment width, and a total addition count according to the fifth embodiment of the present invention; and

FIG. 34 is a flowchart of an example of an operation of a biological-data processing apparatus according to a sixth embodiment of the present invention.

### DESCRIPTION OF THE EMBODIMENTS

In the conventional technology, when performing an addition-averaging process on the biological data measured in response to a plurality of trigger signals, the effect of the addition-averaging process may not be attained equally for each of the stimulated parts due to the difference in the addition count for each stimulated part corresponding to the trigger signal. The configuration of Patent Document 1 discloses the addition-averaging process of biological data according to one trigger signal, and, therefore, such a problem cannot be solved.

A problem to be addressed by an embodiment of the present invention is to equally attain the effect of the addition-averaging process for each of the stimulated parts when the addition-averaging process is performed on a plurality of pieces of biological data measured in response to a trigger signal associated with a stimulus applied to a plurality of parts.

Hereinafter, an embodiment for carrying out the present invention will be described with reference to the drawings. In each drawing, the same elements are denoted by the same reference numerals, and overlapping descriptions may be omitted.

The following embodiments are examples of a biological-data processing apparatus that embodies the technical idea of the present invention, and the present invention is not limited to the following embodiments. Unless otherwise specified, the shape, relative arrangement, parameter values, and the like, of the elements described below are not intended to limit the scope of the present invention, but are intended to be exemplary. Further, the size, the positional relationship, and the like, of the members illustrated in the drawings may be exaggerated for the purpose of clarification.

The biological-data processing apparatus according to an embodiment is an apparatus that performs a process based on the biological data measured by a measurement apparatus. A process based on biological data is, for example, a process to estimate action currents.

The measurement apparatus is a measurement apparatus such as a magnetospinograph that measures, as biological data, the magnetic field generated in response to stimulus such as electrical stimulus. A magnetospinograph is a measurement apparatus that measures a slight spinal cord evoked magnetic field and enables neural activity to be visualized non-invasively (see, for example, Ushio, Shuta et al. “Visualization of the electrical activity of the cauda equina using a magnetospinography system in healthy subjects”, Clinical Neurophysiology, Volume 130, Issue 1, January 2019, pp. 1-11).

The action current is a weak current that flows according to a potential difference that arises when an action potential is generated when cells or tissue of the living body are stimulated, causing the stimulated part to have a negative potential relative to the rest of the living body.

In an embodiment, the addition-average data, which is the result of the addition-averaging process performed every time the addition count of the biological data measured in response to a plurality of trigger signals reaches a predetermined count, and the addition count in the addition-average data, are stored in association with each other.

Then, the processing based on the biological data is performed by using the addition-average data corresponding to the trigger signal acquired by referring to the storage unit based on the specified addition count. A process based on biological data is, for example, a process for estimating the intensity of an action current for each of the one or more stimulated parts.

In an embodiment, by using the addition-average data according to the same addition count for each of the one or more stimulated parts, the difference in the addition count for each stimulated part can be eliminated, and the effect of the addition-averaging process can be attained equally for each stimulated part.

Hereinafter, an embodiment will be described by taking as an example, a biological-data measurement system including a measurement apparatus for measuring a biomagneticfield and a biological-data processing apparatus for estimating an action current from the biological data measured by the measurement apparatus. In the embodiment, an example of estimating the intensity of a current flowing through a nerve in the spinal cord in a living body by applying electrical stimulus to the living body, will be described.

The “user” described above and below is a user using the biological-data measurement system. More specifically, the user may be a technician who acquires biological data by using the biological-data measurement system, or a doctor who performs medical examination or diagnosis.

### EMBODIMENT

### <Overall Configuration of a Biological-Data Measurement System 1>

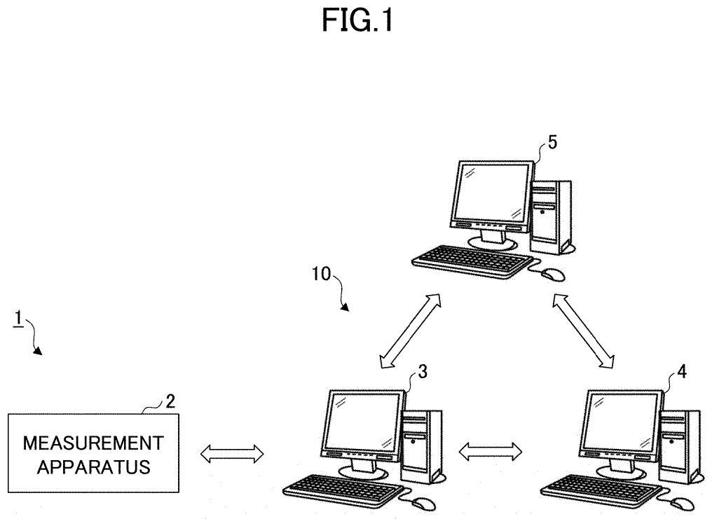

First, the overall configuration of the biological-data measurement system 1 according to the embodiment will be described with reference to FIG. 1.

FIG. 1 is a diagram illustrating an example of the overall configuration of the biological-data measurement system 1. As illustrated in FIG. 1, the biological-data measurement system 1 includes a measurement apparatus 2, a measurement WS (Work Station) 3, an analysis WS 4, and a data storage server 5. These apparatuses are communicatively connected to each other in a wired or wireless manner. Among these, the measurement WS 3, the analysis WS 4, and the data storage server 5 configure a biological-data processing apparatus 10.

The measurement apparatus 2 is a magnetospinograph that measures biomagneticfield data in response to stimulus such as electrical stimulus corresponding to each of a plurality of trigger signals. Biomagneticfield data is an example of measurement data. The measurement apparatus 2 transmits the biomagneticfield data, which is a measurement result with respect to a trigger signal associated with stimulus to each of the plurality of parts, to the measurement WS 3 together with a plurality of trigger signals.

The measurement WS 3 counts a plurality of trigger signals received from the measurement apparatus 2, acquires an addition count for each of the plurality of stimulated parts, and performs addition-averaging processing on the biomagneticfield data every time the addition count for each of the plurality of stimulated parts reaches a predetermined count. Then, the addition-average data that is the result of the addition-averaging process, and information of the addition count in the addition-average data, are associated with each other and transmitted to the data storage server 5.

The data storage server 5 stores, in association with each other, the addition-average data and the addition count, received from the measurement WS 3.

The analysis WS 4 acquires the addition-average data for each of the plurality of stimulated parts by referring to the data storage server 5, based on the addition count specified by the user, and estimates the intensity of the action current for each of the plurality of stimulated parts by using the acquired addition-average data. The analysis WS 4 can display the estimation result, obtained by estimating the intensity of the action current, on the display of the analysis WS 4, transmit the estimation result to the data storage server 5 to be stored, or transmit the estimation result to an external apparatus such as an external server.

The present embodiment illustrates an example of a configuration in which the biological-data processing apparatus 10 is configured by three apparatuses including the measurement WS 3, the analysis WS 4, and the data storage server 5, but the present embodiment not limited thereto. The biological-data processing apparatus 10 may be configured by a single apparatus in which the functions of the measurement WS 3, the analysis WS 4, and the data storage server 5 are integrated, or the biological-data processing apparatus 10 may be configured by four or more apparatuses over which the functions of the measurement WS 3, the analysis WS 4, and the data storage server 5 are distributed.

The biological-data measurement system 1 may include apparatuses other than the measurement WS 3, the analysis WS 4, and the data storage server 5 in a communicable manner, or may include other biological-data measurement apparatuses other than the measurement apparatus 2 in a communicable manner.

### <Configuration Example of the Measurement Apparatus 2>

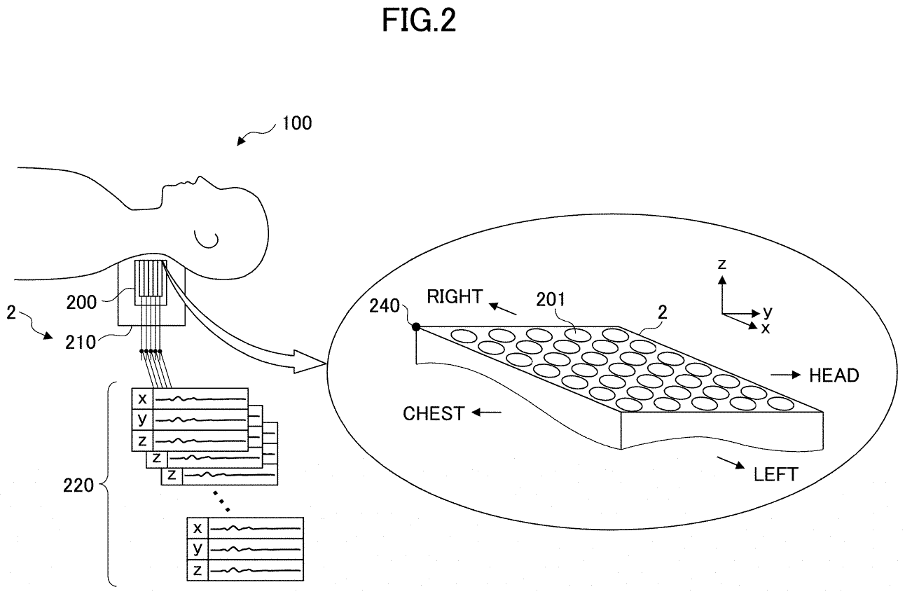

Next, the configuration of the measurement apparatus 2 will be described with reference to FIG. 2.

FIG. 2 is a diagram for explaining an example of a configuration of the measurement apparatus 2. As illustrated in FIG. 2, the measurement apparatus 2 includes a magnetic sensor array 200 and a dewar 210 that houses the magnetic sensor array 200.

The magnetic sensor array 200 is a bio-sensor including a plurality of magnetic sensors 201 arranged in an array and positioned behind the neck of a subject 100. Here, the subject 100 is an example of a “living body”.

Each of the plurality of magnetic sensors 201 measures the biomagneticfield in each direction of an x axis, a y axis, and a z axis illustrated by arrows in FIG. 2 and outputs biomagneticfield data.

In the example of FIG. 2, the magnetic sensor array 200 includes 7×5 magnetic sensors, and the biomagneticfield data measured by each of the plurality of magnetic sensors 201 is output to the biological-data processing apparatus 10. The position where the magnetic sensor array 200 is installed with respect to the subject 100 is adjusted in advance using a marker coil or the like.

The interior of the dewar 210 is filled with liquid helium and is cooled to allow the magnetic sensor array 200 to operate at extremely low temperatures.

In an embodiment, the position of a point 240 on the magnetic sensor array 200 is the origin of the x axis, the y axis, and the z axis. By using the position of the point 240 on the magnetic sensor array 200 as the origin of the x axis, the y axis, and the z axis, the relative positional relationships between the plurality of magnetic sensors 201 in the magnetic sensor array 200 can all be represented by the x, y, and z coordinates.

Further, a known technique described in Japanese Unexamined Patent Application Publication No. 2018-089104 and the like can be applied to the method of measuring the biomagneticfield by the measurement apparatus 2, and, therefore, detailed descriptions thereof will be omitted here.

Further, FIG. 2 illustrates an example where the magnetic sensor array 200 is placed behind the neck of the subject 100, but in the following description, the magnetic sensor array 200 is placed behind the waist of the subject 100 to measure biomagneticfield data near the waist of the living body to estimate the intensity of the current flowing through the nerves in the spinal cord. However, the estimation target is not limited to the intensity of the current flowing through the nerves in the spinal cord. For example, the intensity of current flowing through peripheral nerves in limbs such as arms and feet can be estimated.

### <Example of Hardware Configuration of Computer>

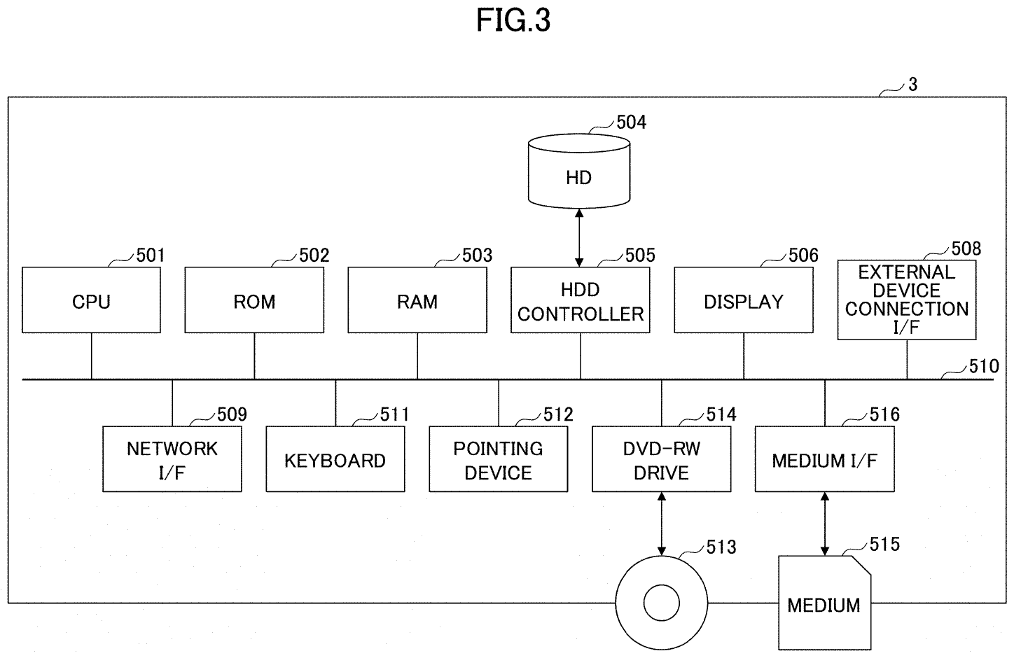

The measurement WS 3, the analysis WS 4, and the data storage server 5 according to the present embodiment can be respectively constructed by a computer. Referring to FIG. 3, the hardware configuration of the computer will be described.

FIG. 3 is a block diagram illustrating an example of a hardware configuration of a computer. FIG. 3 illustrates the hardware configuration of the computer constructing the measurement WS 3. However, the hardware configuration of the computer constructing the analysis WS 4 and the data storage server 5 is the same as that of FIG. 3.

As illustrated in FIG. 3, the measurement WS 3 includes a central processing unit (CPU) 501, a read-only memory (ROM) 502, a random access memory (RAM) 503, a Hard Disk (HD) 504, a Hard Disk Drive (HDD) controller 505, a display 506, an external device connection Interface (I/F) 508, and a network I/F 509.

Further, the measurement WS 3 includes a data bus 510, a keyboard 511, a pointing device 512, a Digital Versatile Disc Rewritable (DVD-RW) drive 514, and a medium I/F 516.

Among these, the CPU 501 controls the operation of the entire measurement WS 3. The ROM 502 stores a program used to drive the CPU 501, such as an Initial Program Loader (IPL).

The RAM 503 is used as the work area of CPU 501. The HD 504 stores various kinds of data such as a program. The HDD controller 505 controls the reading or writing of various kinds of data from or to the HD 504 according to the control of the CPU 501.

The display 506 displays various kinds of information such as cursors, menus, windows, characters, images, or the like. The external device connection I/F 508 is an interface for connecting various external devices. In this case, the external device may be, for example, a Universal Serial Bus (USB) memory, a printer, or the like.

The network I/F 509 is an interface for performing data communication using a network. The data bus 510 is an address bus, a data bus, or the like for electrically connecting elements such as the CPU 501.

The keyboard 511 is a type of input means including a plurality of keys for input of characters, numbers, various instructions, and the like. The pointing device 512 is a type of input means for selecting and executing various instructions, selecting a processing target, moving a cursor, and the like.

The DVD-RW drive 514 controls the reading or writing of various kinds of data from or to a DVD-RW 513 that is an example of a removable recording medium. The recording medium is not limited to a DVD-RW, but may be a Digital Versatile Disc Recordable (DVD-R), etc. The medium I/F 516 controls the reading or writing (storage) of data from or to a recording medium 515, such as a flash memory.

### First Embodiment

### <Example of Functional Configuration of the Biological-Data Processing Apparatus 10>

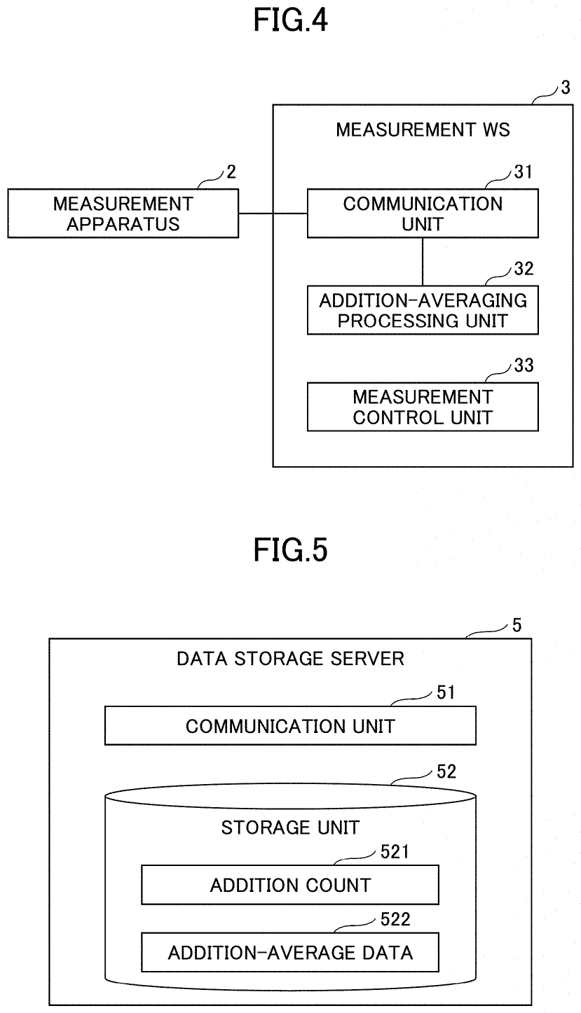

Next, the functional configuration of the measurement WS 3, the analysis WS 4, and the data storage server 5 configuring the biological-data processing apparatus 10 will be described with reference to FIGS. 4 to 6.

### (Example of Functional Configuration of the Measurement WS 3)

First, FIG. 4 is a block diagram illustrating an example of a functional configuration of the measurement WS 3. As illustrated in FIG. 4, the measurement WS 3 includes a communication unit 31, an addition-averaging processing unit 32, and a measurement control unit 33.

Each of these units is a function or functioning means implemented by one of the elements illustrated in FIG. 3 operating in response to an instruction from the CPU 501 according to a program loaded into the RAM 503 from the ROM 502. Although FIG. 4 illustrates the main configuration of the measurement WS 3, the measurement WS 3 may have other configurations. For example, the measurement WS 3 may include a display unit for displaying waveform data representing biomagneticfield data received from the measurement apparatus 2.

The communication unit 31 transmits and receives data and signals to and from the measurement apparatus 2, the analysis WS 4, and the data storage server 5.

The addition-averaging processing unit 32 acquires information of a predetermined count and the total addition count input by a user by using the keyboard 511 (see FIG. 3) or the like. The total addition count is the total number of times that the addition-averaging processing unit 32 adds biomagneticfield data. The addition-averaging processing unit 32 may acquire information of the predetermined count and the total addition count stored in advance in the HD 504 or the like by referring to the HD 504 or the like.

The addition-averaging processing unit 32 receives a plurality of trigger signals from the measurement apparatus 2 via the communication unit 31. The addition-averaging processing unit 32 receives, via the communication unit 31, the biomagneticfield data measured by the measurement apparatus 2 at predetermined intervals from the time when the biomagneticfield measurement is started, with respect to each of the plurality of trigger signals, and performs addition processing. The predetermined intervals may differ for each trigger signal.

The addition-averaging processing unit 32 counts the received plurality of trigger signals and acquires the addition count for each stimulated part corresponding to each trigger signal. The addition-averaging processing unit 32 performs an addition-averaging process on the biomagneticfield data corresponding to each trigger signal, every time the addition count reaches a predetermined count. If the addition count differs for each stimulated part corresponding to each trigger signal, there will be a stimulated part that is subjected to an addition-averaging process and a stimulated part that is not subjected to an addition-averaging process, at the same time.

The addition-averaging process is a process of calculating an average value by dividing, by the addition count, a value obtained by sequentially performing an addition process of adding the biomagneticfield data measured by the measurement apparatus 2. The addition-averaging processing unit 32 associates the addition-average data that is the result obtained by the addition-averaging process and the information on the addition count in the addition-average data with each other, and transmits this associated information to the data storage server 5 via the communication unit 31.

The measurement control unit 33 receives, via the communication unit 31, an instruction based on the estimation result of estimating the intensity of the action current obtained by an estimating unit (described later) of the analysis WS 4, and can cause the measurement apparatus 2 to discontinue or extend the measurement based on the estimation result. The measurement control unit 33 can receive an instruction to discontinue or extend the measurement from the analysis WS 4, as interruption data at any time when the instruction is given.

### (Example of Functional Configuration of the Data Storage Server 5)

Next, FIG. 5 is a block diagram illustrating an example of a functional configuration of the data storage server 5. As illustrated in FIG. 5, the data storage server 5 includes a communication unit 51 and a storage unit 52.

Each of these units is a function or functioning means implemented by one of the elements illustrated in FIG. 3 operating in response to an instruction from the CPU 501 according to a program loaded into the RAM 503 from the ROM 502. Although FIG. 5 illustrates the main configuration of the data storage server 5, the data storage server 5 may have other configurations.

The communication unit 51 transmits and receives data and signals to and from the measurement WS 3 and the analysis WS 4.

The storage unit 52 stores addition-average data 522 received from the measurement WS 3 via the communication unit 51 and an addition count 521 used in the addition-averaging in association with each other. The addition-average data 522 is a generic term of a plurality of pieces of addition-average data. The addition count 521 is a generic term of a plurality of addition counts, and the parameter 523 is a generic term of a plurality of parameters.

### (Example of Functional Configuration of the Analysis WS 4)

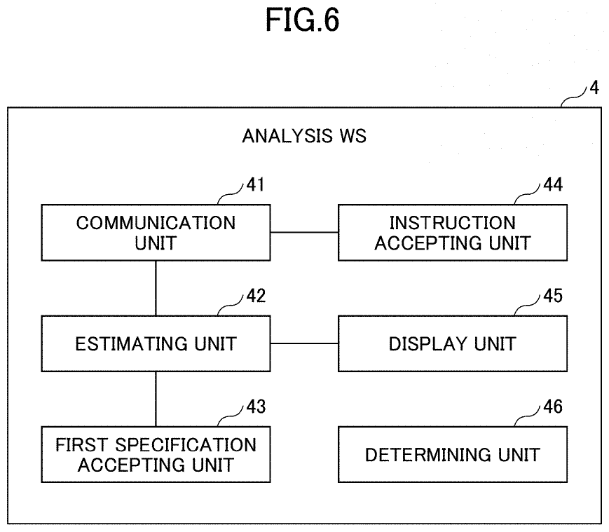

Next, FIG. 6 is a block diagram illustrating an example of a functional configuration of the analysis WS 4. As illustrated in FIG. 6, the analysis WS 4 includes a communication unit 41, an estimating unit 42, a first specification accepting unit 43, an instruction accepting unit 44, a display unit 45, and a determining unit 46.

Each of these units is a function or functioning means implemented by one of the elements illustrated in FIG. 3 operating in response to an instruction from the CPU 501 according to a program loaded into into the RAM 503 from the ROM 502. Although FIG. 6 illustrates the main configuration of the analysis WS 4, the analysis WS 4 may have other configurations.

The communication unit 41 transmits and receives data and signals to and from the measurement WS 3 and the data storage server 5.

The first specification accepting unit 43 accepts information on the addition count that the user has specified by using the keyboard 511 or the like. For example, the first specification accepting unit 43 acquires a list of addition-average data stored in the storage unit 52 via the communication unit 41 and displays a list of acquired addition-average data on the display 506 or the like by the display unit 45. The first specification accepting unit 43 can accept information on the addition count based on the result of the selection made by the user by viewing the list of the addition-average data.

The estimating unit 42 is an example of a biological-data processing unit that performs processing based on biological data. The estimating unit 42 performs a process of estimating the intensity of the action current based on the biomagneticfield data measured by the measurement apparatus 2.

Specifically, the estimating unit 42 acquires the addition-average data for each of a plurality of stimulated parts by referring to the storage unit 52 of the data storage server 5 via the communication unit 41 based on the specified addition count. The storage unit 52 stores the addition count and the addition-average data in association with each other for each of the plurality of stimulated parts of the living body. Therefore, by specifying the addition count, the addition-average data corresponding to the addition count can be acquired for each of the plurality of stimulated parts.

The estimating unit 42 uses the acquired addition-average data to estimate the intensity of the action current for each of a plurality of stimulated parts. As the estimation algorithm, it is possible to use “Array-Gain Constraint Minimum-Norm Spatial Filter With Recursively Updated Gram Matrix” (see, for example, Kumihashi, Isamu et al. “Array-Gain Constraint Minimum-Norm Spatial Filter With Recursively Updated Gram Matrix For Biomagnetic Source Imaging”, IEEE Transactions on Biomedical Engineering, Volume: 57, Issue: 6, June 2010, pp. 1358-1365) or the like.

Here, there may be cases where the measurement cycle of measuring of the biomagneticfield data by the measurement apparatus 2 is different among a plurality of stimulated parts of the living body. Further, there may be cases where a failure occurs in generating a trigger or in measuring, only for the trigger of a particular stimulated part, thereby causing a part of the data to be missing. For this reason, for example, when addition-average data is acquired for each stimulated part based on the measurement time, there are cases where the addition count differs for each stimulated part. If the addition count differs, the effect of the addition-averaging process cannot be attained equally for each stimulated part, and the precision in estimating the intensity of the action current will differ for each stimulated part.

In contrast, in the present embodiment, the effect of the addition-averaging process can be attained equally for each stimulated part, because the intensity of the action current is estimated by using the addition-average data corresponding to the same addition count for each stimulated part. Accordingly, the precision of estimation of the intensity of the action current is the same for each stimulated part.

The display unit 45 displays the estimation result of estimating the intensity of the action current by the estimating unit 42. For example, the display unit 45 may display the estimation result on the display 506 and allow the user to view the result. The display unit 45 can receive, via the measurement WS 3, the waveform data representing the biomagneticfield data measured by the measurement apparatus 2, and display the waveform data as well.

Even in the middle of the measurement, the estimating unit 42 can estimate the intensity of the action current with respect to the data (of the addition count in the middle of the measurement) that is already stored, and the user can view the estimation result obtained by the estimating unit 42 displayed by the display unit 45.

The user may view the estimation result of the intensity of the action current displayed on the display 506 to determine whether the estimation result is valid for each stimulated part, or whether a sufficient amount of the biomagneticfield data has been measured, and so on.

If the user determines that the estimation result of the action current is not valid, the user inputs an instruction to discontinue the measurement by using the keyboard 511 or the like. The instruction accepting unit 44 transmits the accepted discontinuation instruction to the measurement WS 3 via the communication unit 41. The measurement control unit 33 of the measurement WS 3 can cause the measurement apparatus 2 to discontinue the measurement in response to the instruction.

If the user determines that the amount of biomagneticfield data is insufficient, the user inputs an instruction to extend the measurement by using the keyboard 511 or the like. The instruction accepting unit 44 transmits the accepted extension instruction to the measurement WS 3 via the communication unit 41. The measurement control unit 33 of the measurement WS 3 may cause the measurement apparatus 2 to extend the measurement process in response to the instruction.

The determining unit 46 determines whether the estimation result obtained by the estimating unit 42 corresponds to the total addition count and determines whether there is addition-average data corresponding to the total addition count. The determining unit 46 determines whether to extend the measurement. The determining unit 46 outputs a signal instructing to discontinue or extend the measurement to the instruction accepting unit 44 in accordance with the determination results.

The instruction accepting unit 44 may accept not only an instruction from the user to discontinue or to extend the measurement based on the user's determination, but also an instruction from the determining unit 46 to discontinue or to extend the measurement, and transmit the instruction to the measurement WS 3 via the communication unit 41.

### <Example of Operation of the Biological-Data Processing Apparatus 10>

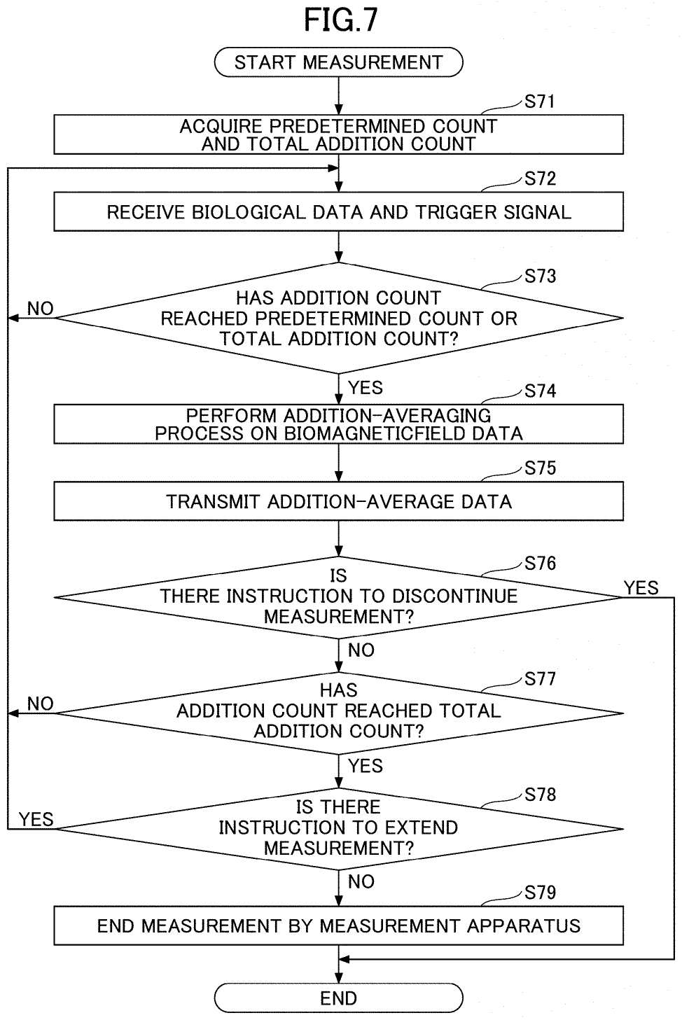

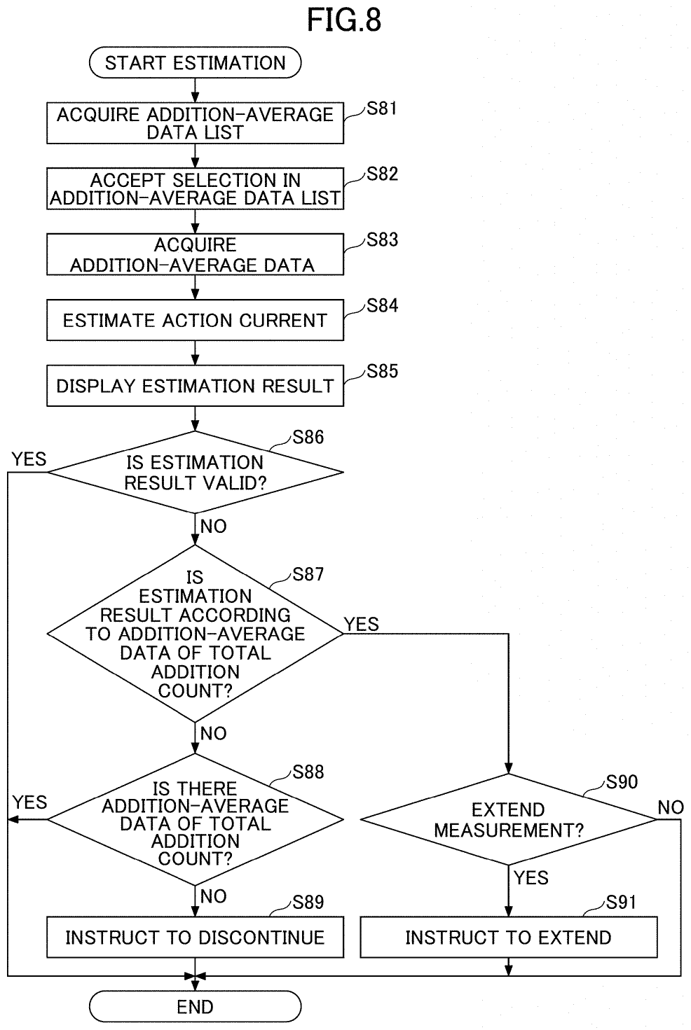

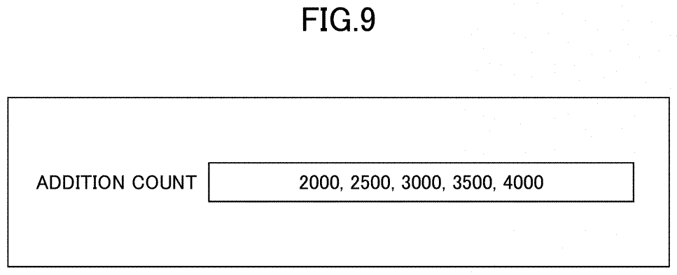

Next, the respective operations of the measurement WS 3 and the analysis WS 4 configuring the biological-data processing apparatus 10 will be described with reference to FIGS. 7 and 8.

### (Example of Operation of the Measurement WS 3)

First, FIG. 7 is a flowchart illustrating an example of the operation of the measurement WS 3. FIG. 7 illustrates the operation of the measurement WS 3 that is triggered at the time point when the biological-data measurement system 1 starts the measurement.

First, in step S71, the addition-averaging processing unit 32 acquires information on a predetermined count and the total addition count that the user inputs by using the keyboard 511 or the like. The addition-averaging processing unit 32 may acquire information on a predetermined count and the total addition count stored in the HD 504 or the like from the HD 504 or the like.

Subsequently, in step S72, the addition-averaging processing unit 32 receives a plurality of trigger signals and biomagneticfield data measured for each of the plurality of stimulated parts corresponding to each of the plurality of trigger signals, from the measurement apparatus 2 via the communication unit 31, and performs an addition process.

Subsequently, in step S73, the addition-averaging processing unit 32 counts the received plurality of trigger signals and acquires the addition count for each of the plurality of stimulated parts, and determines whether the addition count for each of the plurality of stimulated parts has reached the predetermined count.

In step S73, if it is determined that the predetermined count is reached in (YES in step S73), in step S74, the addition-averaging processing unit 32 performs addition-averaging processing on the biomagneticfield data for each of a plurality of stimulated parts. On the other hand, if it is determined that the predetermined count is not reached (NO in step S73), the operations from step S72 and onward are performed again.

Subsequently, in step S74, the addition-averaging processing unit 32 associates the addition-average data, which is the result of the addition-averaging processing, and information on the addition count in the addition-average data with each other, and transmits the associated information to the data storage server 5 via the communication unit 31. The data storage server 5 can store the received addition-average data and the information on the addition count in association with each other.

Subsequently, in step S76, the measurement control unit 33 determines whether an instruction to discontinue the measurement is given.

If it is determined in step S76 that an instruction to discontinue the measurement is given (YES in step S76), the operation proceeds to step S79. On the other hand, if it is determined that an instruction to discontinue the measurement is not given (NO in step S76), the operation proceeds to step S77.

The operation of step S76 is based on interrupt data from the analysis WS 4 and is performed at any timing. Accordingly, operation of step S76 may be performed at any timing from step S71 to step S79.

Subsequently, in step S77, the measurement control unit 33 determines whether the addition count has reached the total addition count. The determination may be made by the addition-averaging processing unit 32 instead of the measurement control unit 33.

If it is determined in step S77 that the total addition count is not reached (NO in step S77), the operations from step S72 and onwards are performed again. On the other hand, in step S77, if it is determined that the total addition count is reached (YES in step S77), in step S78, the measurement control unit 33 determines whether an instruction to extend the measurement has been given.

In step S78, if it is determined that there is an instruction to extend the measurement (YES in step S78), the operations from step S72 and onwards are performed again. On the other hand, if it is determined that there is no instruction to extend the measurement (NO in step S78), in step S79, the measurement control unit 33 ends the measurement in the measurement apparatus 2.

The operation of step S78 is based on interrupt data from the analysis WS 4 and is performed at any timing. Accordingly, the operation of step S78 may be performed at any timing from step S71 to step S79.

In this manner, the measurement WS 3 can perform the addition-averaging process and control the measurement apparatus 2 in response to an instruction to discontinue or extend the measurement.

### (Example of Operation of the Analysis WS 4)

Next, FIG. 8 is a flowchart illustrating an example of an operation of the analysis WS 4. FIG. 8 illustrates the operation of the analysis WS 4 that is triggered at the time point when the user operates the analysis WS 4 to start the estimation process of estimating the action current by using the addition-average data.

First, in step S81, the first specification accepting unit 43 acquires a list of the addition-average data stored in the storage unit 52 via the communication unit 41 and the display unit 45 displays the acquired list of the addition-average data on the display 506 or the like. The user can view the displayed list of the addition-average data and select addition-average data by using the keyboard 511 or the like.

Subsequently, in step S82, the first specification accepting unit 43 accepts information on the addition count based on the addition-average data selected by the user.

Subsequently, in step S83, the estimating unit 42 acquires the addition-average data for each of the plurality of stimulated parts by referring to the storage unit 52 of the data storage server 5 via the communication unit 41 based on the addition count accepted by the first specification accepting unit 43.

Subsequently, in step S84, the estimating unit 42 estimates the intensity of the action current for each of the plurality of stimulated parts by using the acquired addition-average data.

Subsequently, in step S85, the display unit 45 displays the estimation result of estimating the intensity of the action current obtained by the estimating unit 42. For example, the display unit 45 displays the estimation result on the display 506 and allows the user to view the estimation result. Even in the middle of the measurement, the estimating unit 42 can estimate the intensity of the action current with respect to the data (of the addition count in the middle of the measurement) that is already stored, and the user can view the estimation result obtained by the estimating unit 42 displayed by the display unit 45.

Subsequently, in step S86, the instruction accepting unit 44 accepts the determination result of whether the estimation result is valid, by the user who has viewed the estimation result of the intensity of the action current.

In step S86, when a determination result that the estimation result is valid is accepted (YES in step S86), the analysis WS 4 ends the operation. On the other hand, when a determination result that the estimation result is not valid is accepted (NO in step S86), in step S87, the determining unit 46 determines whether the estimation result is based on the addition-average data corresponding to the total addition count. Here, the determination as to whether the estimation result is valid includes a determination as to whether the estimation result is valid as data in the middle of the measurement, in a case of determining by viewing the estimated intensity of the action current by using the data in the middle of the measurement.

If it is determined in step S87 that the estimation result is not based on the addition-average data corresponding to the total addition count (NO in step S87), in step S88, the determining unit 46 determines whether there is addition-average data corresponding to the total addition count.

In step S88, if it is determined that there is addition-average data corresponding to the total addition count (YES in step S88), the analysis WS 4 ends the operation. On the other hand, in step S88, if it is determined that there is no addition-average data corresponding to the total addition count (NO in step S88), in step S89, the instruction accepting unit 44 accepts an instruction to discontinue the measurement from the determining unit 46 and transmits the instruction to the measurement WS 3 via the communication unit 41. Thereafter, the analysis WS 4 ends the operation.

On the other hand, if it is determined in step S87 that the estimation result is based on the addition-average data corresponding to the total addition count (YES in step S87), in step S90, the determining unit 46 determines whether to extend the measurement.

If it is determined in step S90 that the measurement is not to be extended (NO in step S90), the analysis WS 4 ends the operation. On the other hand, if it is determined that the measurement is to be extended (YES in step S90), in step S91, the instruction accepting unit 44 accepts the instruction to extend the measurement from the determining unit 46 and transmits the instruction to the measurement WS 3 via the communication unit 41. Thereafter, the analysis WS 4 ends the operation.

In this manner, the analysis WS 4 can perform the estimation process of estimating the action current and instruct the measurement WS 3 to discontinue or to extend the measurement based on the estimation result.

### <Examples of Various Display Screens>

Next, various display screens displayed by the biological-data measurement system 1 will be described.

### (Example Screen for Specifying the Predetermined Count and the Total Addition Count)

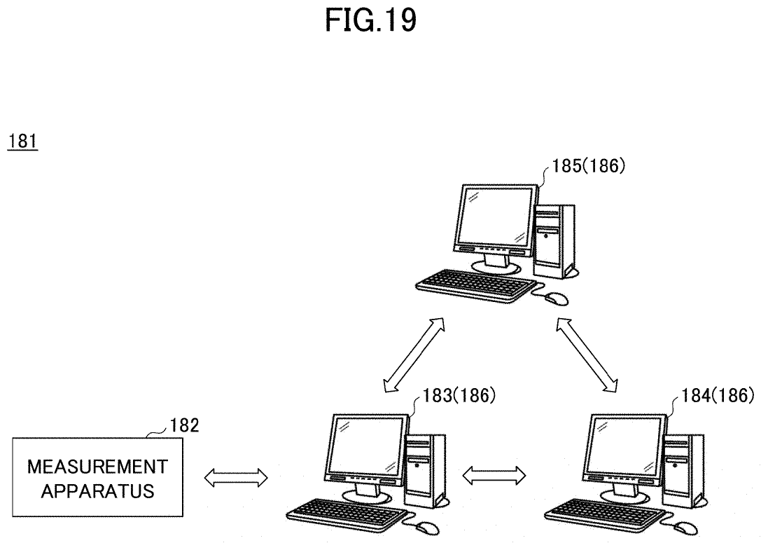

FIG. 19 is a diagram illustrating an example of a screen for specifying a predetermined count and a total addition count displayed by the measurement WS 3. The measurement WS 3 displays the screen illustrated in FIG. 19 on the display when the measurement of biomagneticfield data by the measurement apparatus 2 is started.

In the example illustrated in FIG. 19, 2000 times, 2500 times, 3000 times, and 3500 times each correspond to a predetermined count. Further, 4000 times corresponds to a total addition count.

In the biological-data measurement system 1, an addition-averaging process is performed every time the addition count reaches a predetermined count, and the addition-average data and the addition count in the addition-average data are stored in association with each other in the data storage server 5. Further, every time the addition count reaches the predetermined count, it is possible to estimate the intensity of the action current for each of a plurality of stimulated parts of the living body based on the addition-average data that has undergone the addition-averaging process, and to display the estimation result, so that the user can confirm whether the estimation result is valid in the middle of the measurement.

### (Example of Measurement Screen and Operation Screen)

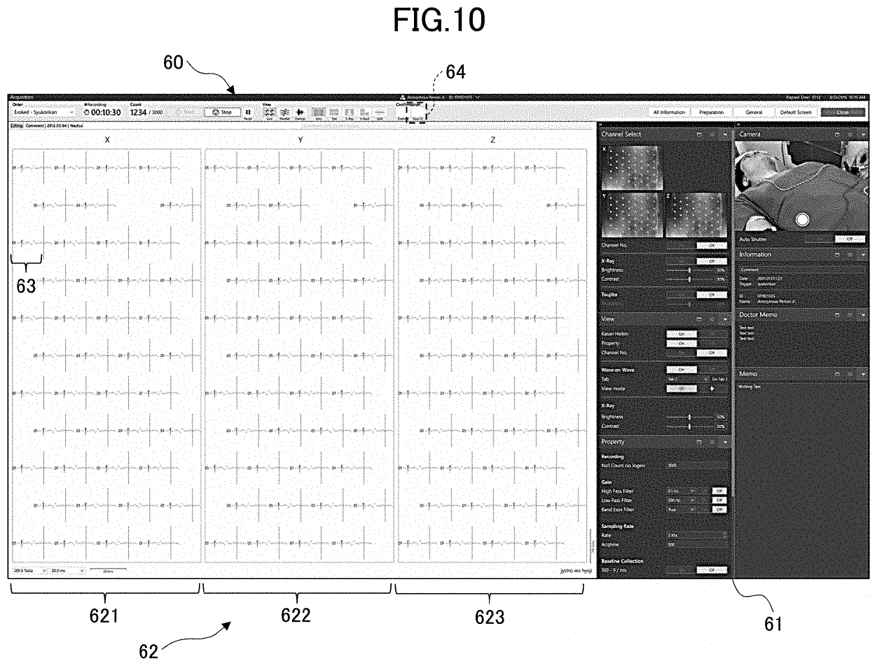

Next, the measurement screen and the operation screen will be described with reference to FIGS. 10 to 14.

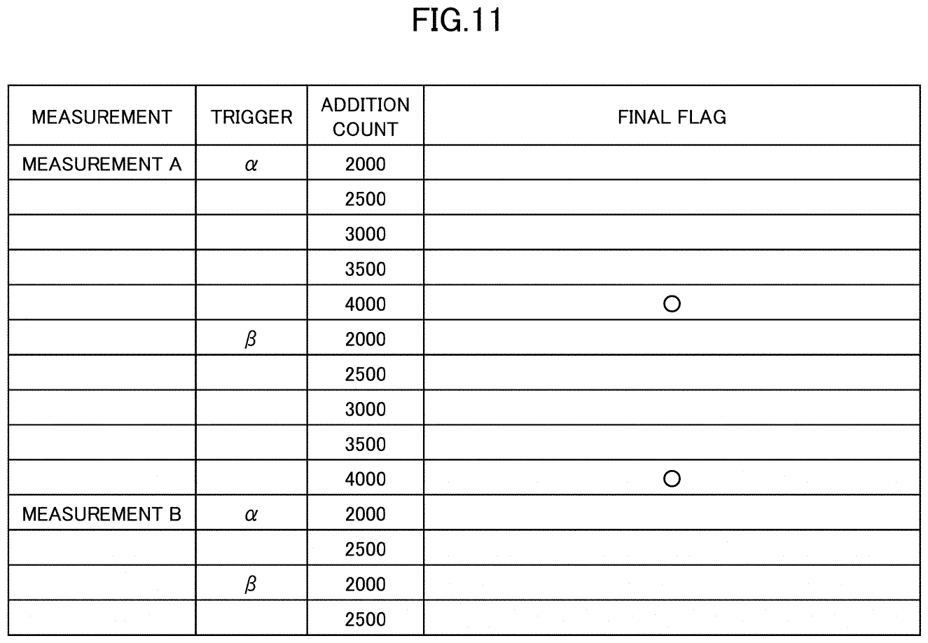

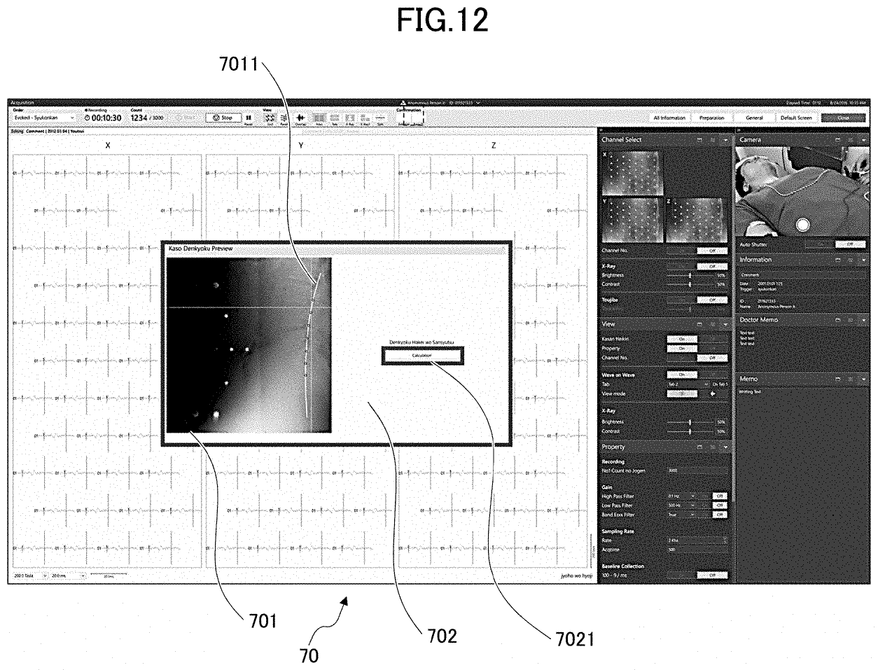

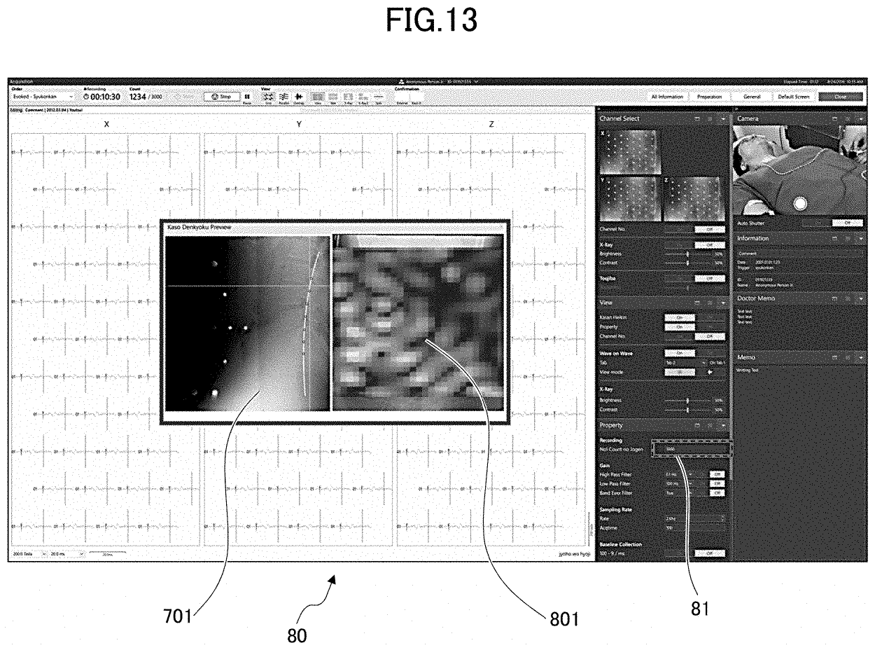

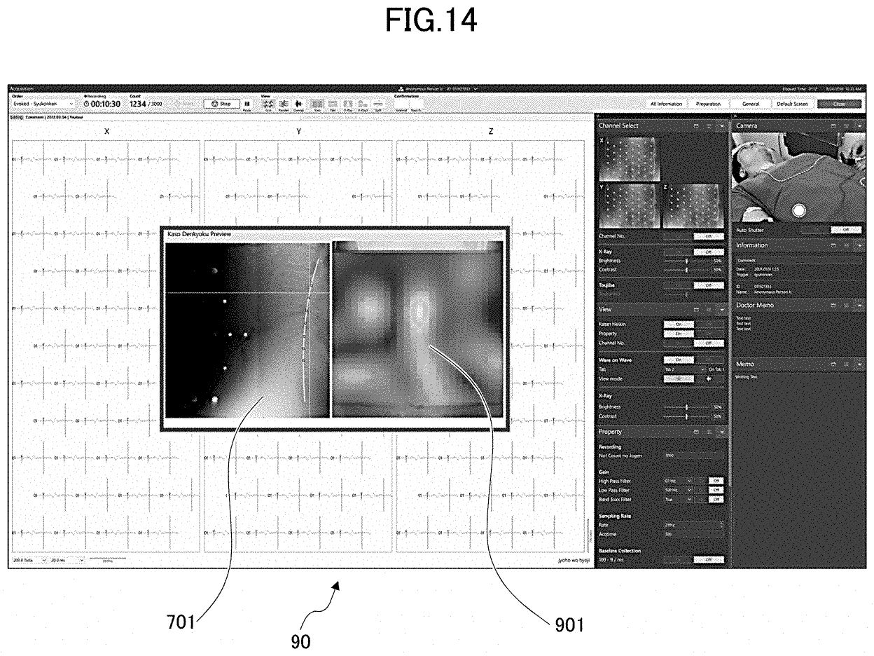

FIG. 10 is a diagram illustrating an example of a measurement screen and an operation screen displayed during measurement by the measurement apparatus 2. FIG. 11 is a diagram illustrating an example of an addition-average data list. FIG. 12 is a diagram illustrating an example of a display screen during action current estimation. FIG. 13 is a diagram illustrating an example of a display screen of an inappropriate estimation result of the action current. FIG. 14 is a diagram illustrating an example of a display screen of an appropriate estimation result of the action current.

As illustrated in FIG. 10, a measurement screen 60 includes an operation screen 61 and a measurement data screen 62. Among these, the operation screen 61 is a screen that is operated by the user to input an instruction to start the measurement, end the measurement, change the display method of the measurement data, or the like.

The measurement data screen 62 displays the biomagneticfield measurement data measured by the measurement apparatus 2. The measurement data screen 62 includes an x measurement data screen 621, a y measurement data screen 622, and a z measurement data screen 623.

The x measurement data screen 621 displays the biomagneticfield data in the x-axis direction of FIG. 2. The y measurement data screen 622 displays biomagneticfield data in the y-axis direction of FIG. 2, and the z measurement data screen 623 displays biomagneticfield data in the z-axis direction of FIG. 2.

Waveform data 63 displayed on the measurement data screen 62 displays biomagneticfield data obtained by one magnetic sensor included in the magnetic sensor array 200. The horizontal axis of the waveform data 63 indicates the time and the vertical axis of the waveform data 63 indicates the biomagneticfield intensity. The waveform data 63 displays, on a real-time basis, the biomagneticfield data obtained by each of a plurality of magnetic sensors included in the magnetic sensor array 200.

The number of pieces of the waveform data 63 included in each of the x measurement data screen 621, the y measurement data screen 622, and the z measurement data screen 623 corresponds to the number of the magnetic sensors included in the magnetic sensor array 200.

Here, the addition-averaging processing unit 32 performs addition-averaging processing on the waveform data 63 acquired in time series.

Specifically, the addition-averaging processing unit 32 acquires the addition-average data by adding the biomagneticfield data per time unit in the waveform data 63 and dividing the addition result of the biomagneticfield data per time unit by the addition count.

Among the data included in the waveform data 63, noise-related data is generated randomly in terms of time, but the noise-related data is canceled out by performing an addition-averaging process. On the other hand, among the data included in the waveform data 63, the biomagneticfield data is accumulated upon being added. Thus, the addition-averaging process can amplify the biomagneticfield data compared to the noise-related data.

The waveform data 63 is an example of biomagneticfield data and is an example of biological data. Waveform data, which is generated by performing an addition-averaging process on the plurality of pieces of the waveform data 63, corresponds to the addition-average data.

In FIG. 10, a start button 64, represented by a dotted line square, is used by the user to give an instruction to start an estimation process of estimating the intensity of the action current by the estimating unit 42. When the user presses the start button 64 by using the cursor of the pointing device 512 in FIG. 3, the instruction accepting unit 44 in the analysis WS 4 sends a request to the data storage server 5 to acquire a list of the addition-average data.

The display unit 45 displays the acquired list of addition-average data on the display 506. FIG. 11 illustrates an example of an addition-average data list. Measurements A and B in FIG. 11 illustrate information indicating the subject as a living body. Triggers α and β represent trigger signals for generating an electrical stimulus to be applied to each of a plurality of stimulated parts in a living body and are examples of multiple trigger signals. The final flag is information indicating the total addition count.

When the user selects an addition count from the displayed addition-average data list, the estimating unit 42 acquires the addition-average data corresponding to the addition count by referring to the storage unit 52 via the communication unit 41 and the communication unit 51. The estimating unit 42 can perform the estimation process by using the addition-average data corresponding to the addition count selected by the user.

Here, after the start button 64 is pressed, a spinal cord position specifying screen 70 as illustrated in FIG. 12 may be displayed. The spinal cord position specifying screen 70 is used to specify different spinal cord positions for each subject. As illustrated in FIG. 12, the spinal cord position specifying screen 70 includes an X-ray image screen 701 and an estimation start instruction reception screen 702.

Among these, the X-ray image screen 701 displays an X-ray image taken from the side of the subject 100 (see FIG. 2). The X-ray image is an image input from an external device via the communication unit 41.

The user can specify a position for estimating the intensity of the action current by viewing the X-ray image screen 701 and specifying a point on the screen by using the cursor of the pointing device 512 of FIG. 3. The intensity of the action current is estimated within the region including the specified position.

In FIG. 12, a curve 7011 included in the X-ray image screen 701 is automatically drawn so that the point specified by the user on the X-ray image screen 701 is included, and corresponds to the position of the spinal cord inside the subject 100 viewed from the side.

After specifying a point on the curve 7011, the user can press the start button 7021 in the estimation start instruction reception screen 702 by using the cursor of the pointing device 512 of FIG. 3, to start the process of estimating the intensity of the action current at a position inside the subject 100 corresponding to the specified point.

Next, as illustrated in FIG. 13, an estimation result display screen 80 includes the X-ray image screen 701 and a distribution diagram 801 of the intensity of the action current. The distribution diagram 801 is a diagram in which a two-dimensional distribution of the estimated intensity of the action current is replaced by colors and displayed, based on the data measured by each magnetic sensor included in the magnetic sensor array 200.

The biological-data measurement system 1 acquires the distribution diagram 801 in time series and displays the distribution diagram 801 in time series to visualize the current flowing through the spinal cord as a video.

In the case of FIG. 13, it can be seen that in the distribution diagram 801, a strong action current is estimated at a location where an action current inside is not supposed be present, that is, at a location outside the human body. This indicates that noise has a significant impact on the estimated intensity of the action current. That is, FIG. 13 illustrates a case in which the estimation result of the action current is inappropriate, because the action current estimated based on the measurement data of the biomagneticfield that includes a lot of noise, because the number of pieces of measurement data used for processing by the addition-averaging processing unit 32 is insufficient.

On the other hand, in the case of FIG. 14, it can be seen that in a distribution diagram 901, a strong action current is estimated at a location where the action current is supposed to be located, that is, on the spinal cord of the human body. This indicates that the effect of noise is reduced in the estimation result of the intensity of the action current.

That is, FIG. 14 illustrates a case where the estimation result is appropriate, because the action current is estimated based on the measurement data of the biomagneticfield in which noise is reduced, because the number of pieces of measurement data used for processing by the addition-averaging processing unit 32 is sufficient.

The user can view the distribution diagram illustrated in FIGS. 13 and 14 to determine whether the estimation result is appropriate. If it is determined that the estimation result is not appropriate, the user operates the start button 64 (see FIG. 10) to instruct the start of the estimation process again.

Instead of accepting the start instruction, the number of pieces of measurement data may be input as a numerical value into an edit box 81 represented by a square of a chain line in FIG. 13, and when the number of pieces of measurement data corresponding to the input number is acquired, the estimation processing by the estimating unit 42 may be started.

<Effect of the Biological-Data Processing Apparatus 10>

As described above, in the embodiment, the addition-averaging processing unit 32 performs an addition-averaging process every time the addition count of biological data that is measured in response to a trigger signal associated with a stimulus corresponding to at least one part, reaches a predetermined count. The storage unit 52 stores the addition-average data, which is the result of the addition-averaging process for each of one or more stimulated parts, and the addition count in the addition-average data, in association with each other. The estimating unit 42 performs processing based on the biological data by using the addition-average data for each of the one or more stimulated parts acquired by referring to the storage unit 52 based on the specified addition count. For example, the estimating unit 42 performs processing based on biological data by estimating the intensity of the action current for each of the one or more stimulated parts.

Thus, by using the addition-average data for which the addition count is the same, for each of the one or more stimulated parts, the difference in the addition count for each stimulated part can be eliminated, and the effect of the addition-averaging process can be attained equally for each stimulated part.

According to the present embodiment, the measurement control unit 33 is provided for controlling the discontinuation or the extension of the measurement by the measurement apparatus 2 based on the estimation result of estimating the intensity of the action current by the estimating unit 42. Further, there is provided the display unit 45 for displaying the estimation result of estimating the intensity of the action current by the estimating unit 42 and the instruction accepting unit 44 for accepting an instruction to either discontinue or extend the measurement by the measurement apparatus 2. The measurement control unit 33 causes the measurement apparatus 2 to discontinue or extend the measurement in accordance with the instruction accepted by the instruction accepting unit 44.

Accordingly, the user can determine whether the estimation result is valid or whether it is necessary to extend the measurement, in the middle of the measurement, and the measurement by the measurement apparatus 2 can be discontinued or extended according to the determination result of the user.

That is, the user may be prompted to confirm and determine, in the middle of the measurement, as to whether the estimation result is valid or whether it is necessary to extend the measurement, and the measurement by the measurement apparatus 2 may be discontinued or extended according to the confirmation result.

Accordingly, it is possible to reduce unnecessary measurement and to extend the measurement according to need.

### Second Embodiment

Next, a biological-data measurement system 1a according to the second embodiment will be described. The same elements as those described in the first embodiment are denoted by the same reference numerals, and overlapping descriptions thereof are omitted accordingly. The same applies to the following embodiments.

According to the present embodiment, the estimating unit switches the estimation method according to the addition count, so that a high estimation precision can be ensured. Specifically, when the addition count does not correspond to the total addition count, the estimating unit performs a faster process than when the addition count corresponds to the total addition count, and when the addition count corresponds to the total addition count, the estimating unit performs a process with higher precision than when the addition count is other than the addition count corresponding to the total addition count.

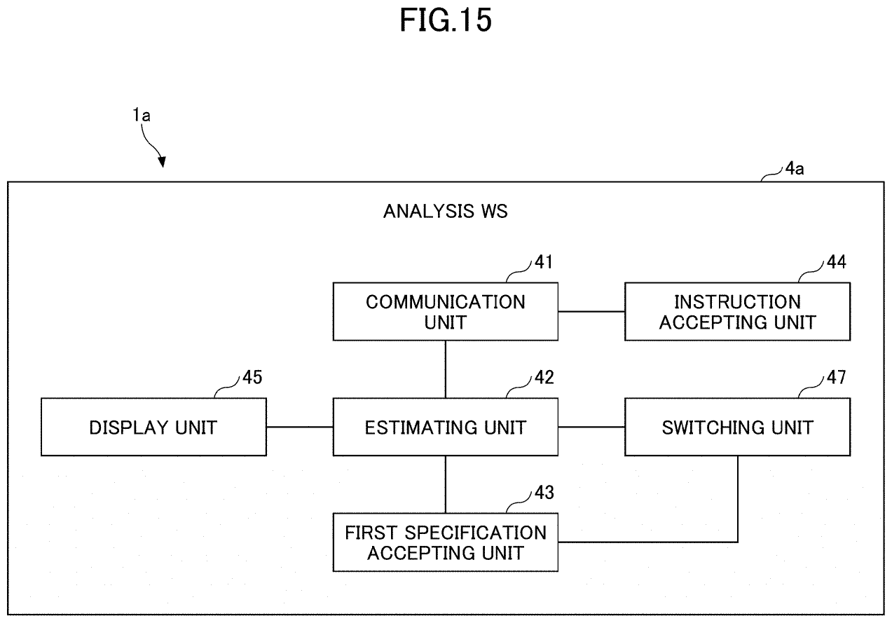

FIG. 15 is a block diagram illustrating an example of the functional configuration of an analysis WS 4a included in the biological-data measurement system 1a. As illustrated in FIG. 15, the analysis WS 4a includes a switching unit 47.

The switching unit 47 switches the estimation processing method of estimating the action current by the estimating unit 42 in accordance with the specified addition count accepted by the first specification accepting unit 43.

For example, the above-described “Array-Gain Constraint Minimum-Norm Spatial Filter With Recursively Updated Gram Matrix” can be applied to the estimation processing method, and the switching of the estimation processing method involves the switching of the number of times of repeating the estimation process. The estimation processing method is an example of a processing method by a biological data processing unit. By associating the specified addition count with the number of repeats in advance, the switching unit 47 can switch the number of repeats according to a specified addition count.

As described above, according to the present embodiment, the estimation processing method is switched by the estimating unit 42 in accordance with the addition count, so that high estimation precision can be ensured. In a case of not increasing the precision, the estimation can be performed at high speed by switching the estimation method by the estimating unit.

### Third Embodiment

Next, a biological-data measurement system 1b according to the third embodiment will be described.

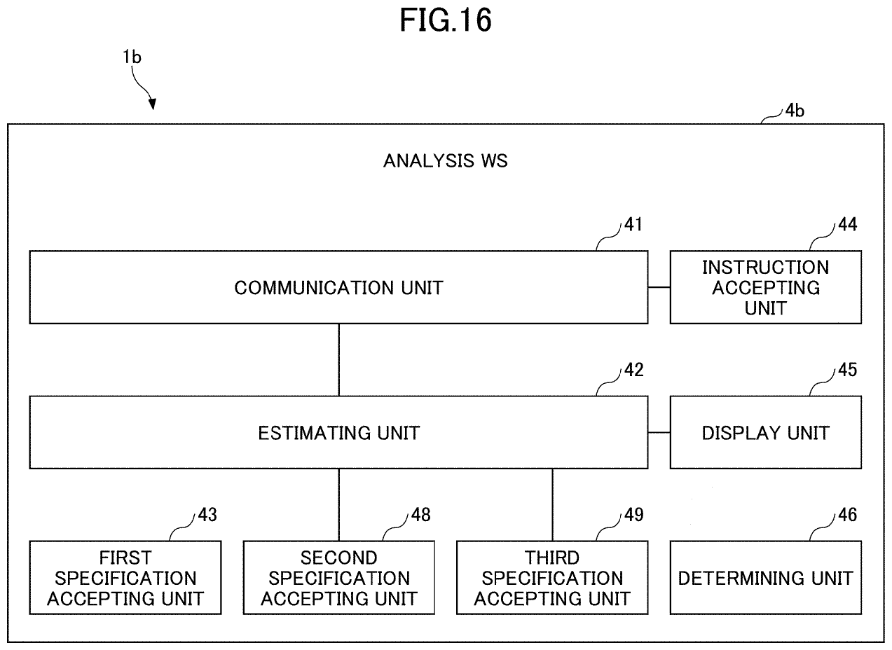

FIG. 16 is a block diagram illustrating an example of the functional configuration of an analysis WS 4b included in the biological-data measurement system 1b. As illustrated in FIG. 16, the analysis WS 4b includes a second specification accepting unit 48 for accepting a specification of a predetermined count and a third specification accepting unit 49 for accepting a specification of an update interval count used for automatically updating the addition count.

The analysis WS 4b accepts a next addition count as the predetermined count by the second specification accepting unit 48. Every time the addition count reaches the predetermined count, the analysis WS 4b can update the predetermined count according to the next addition count accepted by the second specification accepting unit 48. Further, every time the addition count reaches the predetermined count, the analysis WS 4b automatically updates the predetermined count to an addition count obtained by adding the update interval count accepted by the third specification accepting unit 49 to the predetermined count.

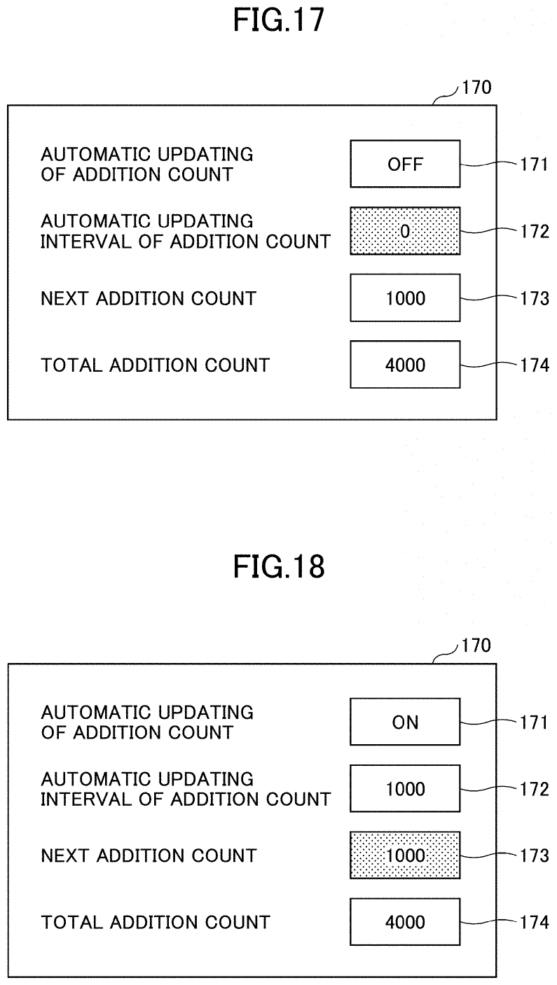

FIGS. 17 and 18 are diagrams illustrating an example of a specification screen 170 for specifying a predetermined count and a total addition count according to the present embodiment, wherein FIG. 17 is a first example, and FIG. 18 is a second example. The specification screen 170 is a graphical user interface (GUI) displayed on the display 506 or the like, and is operated to input information by using the keyboard 511, the pointing device 512, or the like.

As illustrated in FIGS. 17 and 18, the specification screen 170 includes a switch 171, a first input box 172, a second input box 173, and a third input box 174.

The switch 171 is a switch for switching between a setting of performing (ON) and a setting of not performing (OFF) the automatic updating of the addition count.

The first input box 172 is a box for inputting an “automatic updating interval of the addition count”, referring to the update interval count. The third specification accepting unit 49 in FIG. 16 accepts the update interval count via the first input box 172.

The second input box 173 is a box for inputting the next addition count. The second specification accepting unit 48 in FIG. 16 accepts the next addition count as the predetermined count via the second input box 173.

The third input box 174 is a box for inputting the total addition count.

In FIG. 17, the switch 171 is set not to perform the automatic updating of the addition count (OFF). The first input box 172 has been disabled because automatic updating is not performed. Note that the dot hatching in the first input box 172 of FIG. 17 indicates disabled.

In the second input box 173, “1000 times” is input as the next addition count. The analysis WS 4b can specify the predetermined count to any count, for example, by accepting an input of 1500 times as the next addition count, after the addition count reaches 1000.

In FIG. 18, the switch 171 is set to perform the automatic updating of the addition count (ON). The automatic updating is to be performed, and, therefore, “1000 times” is input in the first input box 172 as the automatic updating interval of the addition count (the update interval count), and the second input box 173 is disabled. Note that the dot hatching in the second input box 173 of FIG. 18 indicates disabled.

The analysis WS 4b updates the predetermined count by adding “1000 times” that is the update interval count to the predetermined count every time the addition count reaches the predetermined count. In FIG. 11, 4000 times is specified as the total addition count, and, therefore, every time the addition count reaches the predetermined count, the predetermined count varies from 1000 times to 2000 times, 3000 times, and 4000 times by the automatic updating.

As described above, the biological-data measurement system 1b includes the second specification accepting unit 48 and the third specification accepting unit 49, and, therefore, the user of the biological-data measurement system 1b can appropriately select the method of specifying the predetermined count according to the status of measurement by the biological-data measurement system 1b. As a result, the biological-data measurement system 1b can improve convenience.

### Fourth Embodiment

Next, a biological-data measurement system 181 according to the fourth embodiment will be described.

In the biological-data measurement system, it is preferable to detect a temporary drop in biological signals, or noise mixed in the data, during the measurement.

For example, according to the method described in Japanese Unexamined Patent Application Publication No. 2019-162253, it is possible to detect and exclude sudden large noise, but it is not possible to detect and exclude small noise that is not obvious unless an addition-averaging process is performed. Further, it is not possible to detect and exclude a decrease in biological signals. For example, when some kind of abnormality occurs during the measurement, it is difficult to detect this abnormality with high precision, resulting in an insufficient result for ensuring the quality of the final addition-average data.

According to the present embodiment, when an abnormality is detected during measurement by the biological-data measurement system 181, and data in the section where the abnormality had occurred is excluded, thereby ensuring the reliability of the measurement while ensuring sufficient signal quality.

### <Overall Configuration of the Biological-Data Measurement System 181>

FIG. 19 is a diagram illustrating an example of the overall configuration of the biological-data measurement system 181. The biological-data measurement system 181 includes a measurement apparatus 182, a measurement WS 183, an analysis WS 184, and a data storage server 185. These apparatuses are communicatively connected to each other in a wired or wireless manner. Among these, the measurement WS 183, the analysis WS 184, and the data storage server 185 configure a biological-data processing apparatus 186.

The measurement apparatus 182 is a magnetospinograph that measures the biomagneticfield data of a biomagneticfield generated at a plurality of parts of a living body in response to stimulus such as electrical stimulus according to each of a plurality of trigger signals. Biomagneticfield data is an example of measurement data. The measurement apparatus 182 transmits biomagneticfield data, which is a measurement result of measuring each of a plurality of parts, to the measurement WS 183 together with a plurality of trigger signals corresponding to each of a plurality of parts.

The measurement WS 183 counts the trigger signals received from the measurement apparatus 182, acquires the addition count, and performs the addition-averaging process on the biomagneticfield data every time the addition count reaches a predetermined count. The addition-averaging process is only performed on the biomagneticfield data corresponding to a count that is obtained by counting back from the predetermined count by a count that is specified separately. The count specified separately is referred to as a segment width, and the obtained data is referred to as segment addition data.

The measurement apparatus 182 associates the segment addition data with the segment width, the addition-averaging count (number of times of performing addition-averaging) at the start point of the segment width, and the addition-averaging count of the end point with each other, and transmits the associated information to the data storage server 5. Hereinafter, information including at least one of the three types of information related to the above segment addition will be described as information related to the segment addition.

The data storage server 185 stores the addition-average data received from the measurement WS 183 in association with information related to segment addition.

The analysis WS 184 acquires all segment addition data by referring to the data storage server 185, excludes incorrect segment addition data from the acquired segment addition data, and performs addition-averaging on all segment addition data to obtain the addition-average data. The analysis WS 184 estimates the intensity of the action current based on the obtained addition-average data. The analysis WS 184 can display the estimation result on the display of the analysis WS 184, transmit the estimation result to the data storage server 185 to be stored, or transmit the estimation result to an external device such as an external server.

The present embodiment illustrates an example of a configuration in which the biological-data processing apparatus 186 is configured by three apparatuses including the measurement WS 183, the analysis WS 184, and the data storage server 185, but the present embodiment not limited thereto. The biological-data processing apparatus 186 may be configured by a single apparatus in which the functions of the measurement WS 183, the analysis WS 184, and the data storage server 185 are integrated, or the biological-data processing apparatus 186 may be configured by four or more apparatuses over which the functions of the measurement WS 183, the analysis WS 184, and the data storage server 185 are distributed.

The biological-data measurement system 181 may include apparatuses other than the measurement WS 183, the analysis WS 184, and the data storage server 185 in a communicable manner, or may include other biological-data measurement apparatuses other than the measurement apparatus 182 in a communicable manner.

The configuration of the measurement apparatus 182 is similar to the configuration of the measurement apparatus 2 described with reference to FIG. 2 in the first embodiment. The hardware configuration of a computer is similar to the hardware configuration of the computer described with reference to FIG. 3 in the first embodiment.

### <Example of Functional Configuration of the Biological-Data Processing Apparatus 186>

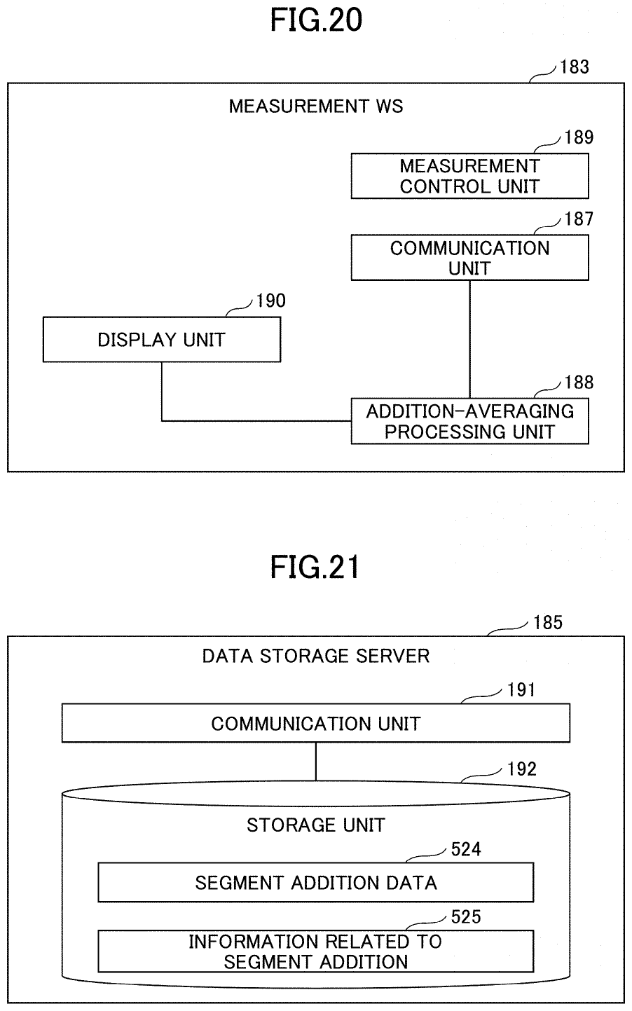

Referring to FIGS. 20, 21, and 22, the functional configuration of the measurement WS 183, the analysis WS 184, and the data storage server 185 configuring the biological-data processing apparatus 186 will be described.

### (Example of Functional Configuration of the Measurement WS 183)

FIG. 20 is a block diagram illustrating an example of a functional configuration of a measurement WS 183. The measurement WS 183 includes a communication unit 187, an addition-averaging processing unit 188, a measurement control unit 189, and a display unit 190.

Each of these units is a function or functioning means implemented by one of the elements illustrated in FIG. 3 operating in response to an instruction from the CPU 501 according to a program loaded into the RAM 503 from the ROM 502. Although FIG. 20 illustrates the main configuration of the measurement WS 183, the measurement WS 183 may have other configurations.

The communication unit 187 transmits and receives data and signals to and from the measurement apparatus 182, the analysis WS 184, and the data storage server 185.

The addition-averaging processing unit 188 acquires information on a predetermined count, a segment width, and a total addition count that the user inputs by using the keyboard 511 (see FIG. 3) or the like. The total addition count is the total number of times that the addition-averaging processing unit 188 adds biomagneticfield data. The addition-averaging processing unit 188 may acquire information on a predetermined count, a segment width, and a total addition count stored in advance in the HD 504 or the like by referring to the HD 504 or the like.

The addition-averaging processing unit 188 receives at least one trigger signal from the measurement apparatus 182 via the communication unit 187. Further, in response to at least one trigger signal, the addition-averaging processing unit 188 receives, via the communication unit 187, biomagneticfield data measured by the measurement apparatus 182 corresponding to a count that is obtained by counting back from the predetermined count by the segment width, and performs an addition-averaging process on the received biomagneticfield data.

The addition-averaging process is a process for calculating an average value by dividing, by the addition count, a value obtained by performing an addition process on the biomagneticfield data measured by the measurement apparatus 182 corresponding to a count that is obtained by counting back from the predetermined count by the segment width. The addition-averaging processing unit 188 transmits segment addition data that is the processing result to the display unit. The addition-averaging processing unit 188 associates the segment addition data with the information related to the segment addition, and transmits the associated information to the data storage server 185 via the communication unit 187.

The display unit 190 displays the segment addition data as waveform data on the display. When displaying the segment addition data, the display unit 190 may display, in parallel, the segment addition data associated with a plurality of predetermined counts, as waveform data. Further, the display unit 190 may display a frequency spectrum or the like which is the result of analysis of the segment addition data performed by some means. The measurement WS 183 evaluates the displayed waveform data, determines whether the measurement has been performed normally in the segment, and records the determination result.

The measurement WS 183 determines whether the measurement has been performed normally in the segment according to a predetermined algorithm. However, the user may visually determine whether the measurement has been performed normally in the segment. For example, the user can view the frequency spectrum and determine that there is an abnormality when there is a peak at a position other than the biological signal in the frequency, or determine that there is an abnormality when the amplitude of the biological signal obtained from the waveform data is obviously lowered in comparison to the amplitude of the other segment addition data. “Obviously lowered” means that the amplitude is lowered by an extent greater than or equal to the noise level included in the segment addition data, for example.

If it is determined that the segment addition data is abnormal data, the measurement WS 183 increases the addition count by an amount corresponding to the segment width, to exclude this abnormal segment addition data when generating the addition-average data. For example, the measurement WS 183 accepts an instruction to extend the measurement given by a user by using the keyboard 511 or the like and increases the addition count by an amount corresponding to the segment width. The measurement control unit 189 may cause the measurement apparatus 182 to extend the measurement in response to the extension instruction.

The measurement control unit 189 receives an instruction based on the evaluation result of evaluating the waveform data displayed on the display unit 190, via the communication unit 187, and causes the measurement apparatus 182 to discontinue or extend the measurement, based on the evaluation result. The measurement control unit 189 can receive the instruction to discontinue or extend the measurement as interrupt data at any timing when the instruction is given.

### (Example of Functional Configuration of the Data Storage Server 185)

FIG. 21 is a block diagram illustrating an example of a functional configuration of the data storage server 185. The data storage server 185 includes a communication unit 191 and a storage unit 192.

Each of these units is a function or functioning means implemented by one of the elements illustrated in FIG. 3 operating in response to an instruction from the CPU 501 according to a program loaded into the RAM 503 from the ROM 502. Although FIG. 21 illustrates the main configuration of the data storage server 185, the data storage server 185 may have other configurations.

The communication unit 191 transmits and receives data and signals to and from the measurement WS 183 and the analysis WS 184.

The storage unit 192 stores segment addition data 524 received from the measurement WS 183 via the communication unit 191 and information related to segment addition 525, in association with each other. The segment addition data 524 is a generic term of a plurality of pieces of segment addition data, and the information related to segment addition 525 is a generic term of the information related to a plurality of segment additions.

### (Example of Functional Configuration of the Analysis WS 184)

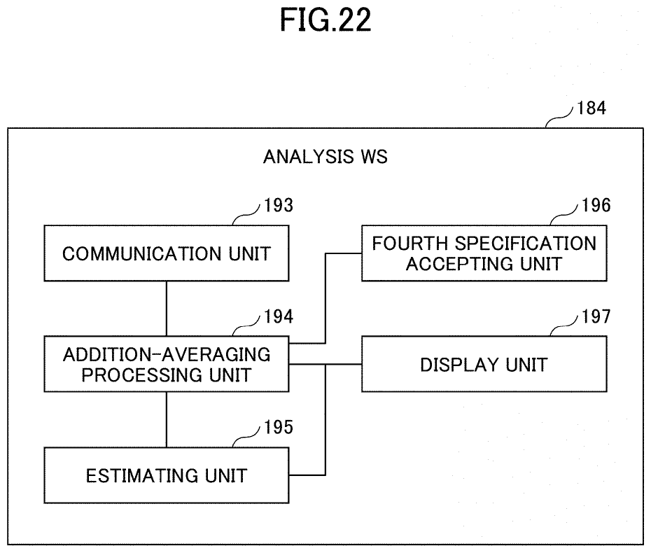

Next, FIG. 22 is a block diagram illustrating an example of a functional configuration of the analysis WS 184. As illustrated in FIG. 22, the analysis WS 184 includes a communication unit 193, an addition-averaging processing unit 194, an estimating unit 195, a fourth specification accepting unit 196, and a display unit 197.

Each of these units is a function or functioning means implemented by one of the elements illustrated in FIG. 3 operating in response to an instruction from the CPU 501 according to a program loaded into the RAM 503 from the ROM 502. Although FIG. 22 illustrates the main configuration of the analysis WS 184, the analysis WS 184 may have other configurations.

The communication unit 193 transmits and receives data and signals to and from the measurement WS 183 and the data storage server 185.

The addition-averaging processing unit 194 acquires a list of segment addition data stored in the storage unit 192 via the communication unit 193 and displays the acquired list of segment addition data on the display 506 or the like by the display unit 197. The fourth specification accepting unit 196 can accept information on the addition count based on the result of the selection made by the user by viewing the list of the segment addition data. At least one piece of segment addition data selected is subjected to an addition-averaging process based on the addition count of each piece of data. The analysis WS 184 can exclude abnormal segment addition data from the list of segment addition data presented, and acquire addition-average data.

The estimating unit 195 is an example of a biological-data processing unit that performs processing based on biological data. The estimating unit 195 performs the process of estimating the intensity of the action current, based on the biomagneticfield data generated by the addition-averaging processing unit 194. The estimating unit 195 estimates the intensity of the action current for each of a plurality of parts by using the acquired addition-average data. As the estimation algorithm, it is possible to use “Array-Gain Constraint Minimum-Norm Spatial Filter With Recursively Updated Gram Matrix” or the like described above.

The display unit 197 displays the estimation result of estimating the intensity of the action current by the estimating unit 195. For example, the display unit 197 may display the estimation result on the display 506 and allow the user to view the result. The display unit 197 can display the waveform data indicated the acquired addition-average data and segment addition data.

The user may view the estimation result of the intensity of the action current displayed on the display 506 to determine whether the measurement has been performed normally in the segment. If the user determines that the segment addition data is abnormal data, the analysis WS 184 excludes the segment addition data when generating the addition-average data.

### <Example of Operation of the Biological-Data Processing Apparatus 186>

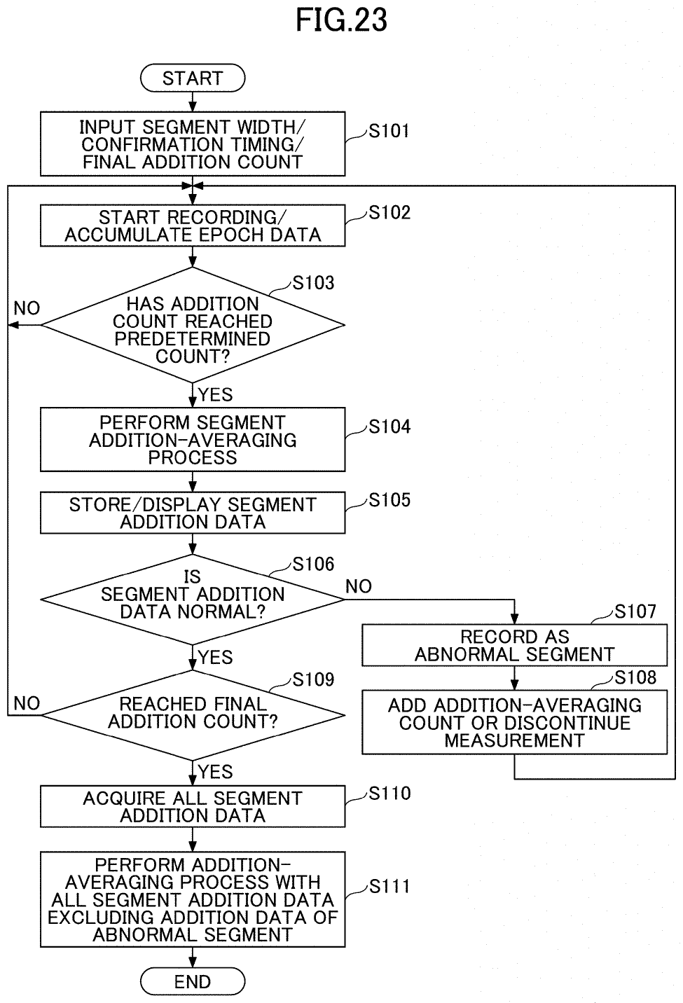

Next, the operation of the biological-data processing apparatus 186 will be described with reference to FIG. 23. FIG. 23 is a flowchart illustrating an example of the operation of the biological-data processing apparatus 186. The biological-data processing apparatus 186 starts the operation of FIG. 23, upon accepting an instruction by a user to start biological-data processing and the like.

First, in step S101, the biological-data processing apparatus 186 acquires, by the addition-averaging processing unit 188, information on a predetermined count, a segment width, and a total addition count that are input by a user by using the keyboard 511 or the like. The addition-averaging processing unit 188 may acquire, from the HD 504 or the like, information on the predetermined count, the segment width, and the total addition count stored in the HD 504 or the like in advance.

Subsequently, in step S102, the biological-data processing apparatus 186 receives, by the addition-averaging processing unit 188, at least one trigger signal and biomagneticfield data measured in association with this trigger signal, from the measurement apparatus 182 via the communication unit 187, and stores the received information. In the description of the present embodiment, one piece of biomagneticfield data corresponding to one trigger signal is referred to as epoch data.

Subsequently, in step S103, the biological-data processing apparatus 186 causes the addition-averaging processing unit 188 to count the received trigger signal and acquire the addition count with respect to each trigger, and determines whether the addition count has reached a predetermined count.

If it is determined that the addition count has reached a predetermined count (YES in step S103), in step S104, the biological-data processing apparatus 186 performs, by the addition-averaging processing unit 188, addition-averaging processing on the biomagneticfield data corresponding to a count that is obtained by counting back from the predetermined count by the segment width. On the other hand, if it is determined that the addition count has not reached a predetermined count (NO in step S103), the biological-data processing apparatus 186 performs the operation of step S102 again.

Subsequently, in step S105, the biological-data processing apparatus 186 causes the addition-averaging processing unit 188 to associate the segment addition data, which is the result of the segment addition-averaging processing, and the information on the addition count in the segment addition data with each other, and transmits the associated information to the data storage server 185 via the communication unit 187. The data storage server 185 stores the received segment addition data and the information related to the segment addition in association with each other. The biological-data processing apparatus 186 displays the segment addition data on the display unit 190 of the measurement WS 183.

Subsequently, in step S106, the biological-data processing apparatus 186 confirms the segment addition data displayed on the display unit 190 by the measurement WS 183, and determines whether the segment addition data is normal. For example, the measurement WS 183 may determine whether the segment addition data is normal by accepting an operation from a user input by using the keyboard 511 or the like.

In step S106, if it is determined that the segment addition data is not normal (NO in step S106), in step S107, the biological-data processing apparatus 186 records this segment as an abnormal segment by the measurement WS 183.

Subsequently, in step S108, the biological-data processing apparatus 186 causes the measurement WS 183 to increase the final addition count, that is, add the addition-averaging count to immediately update the final addition count, or to discontinue the measurement. Thereafter, the biological-data processing apparatus 186 shifts to the operation of step S102.

Note that the biological-data processing apparatus 186 may perform the operation of step S108 at any timing from step S101 to step S107. The biological-data processing apparatus 186 may similarly cause the measurement WS 183 to update the settings of the segment and the predetermined count upon receiving an instruction at any timing.

Subsequently, in step S109, the biological-data processing apparatus 186 determines, by the measurement control unit 189, whether the addition count has reached the final addition count (the total addition count). The determination may be made by the addition-averaging processing unit 188 instead of the measurement control unit 189.

In step S109, if it is determined that the final addition count is not reached (NO in step S109), the biological-data processing apparatus 186 performs the operation from step S102 again. On the other hand, in step S109, if it is determined that the final addition count is reached (YES in step S109), the biological-data processing apparatus 186 causes the measurement apparatus 182 to end the measurement.

In this manner, the biological-data processing apparatus 186 can execute the addition-averaging process by the measurement WS 183 and control the measurement apparatus 2 in response to an instruction to discontinue or extend the measurement.

Subsequently, in step S110, the biological-data processing apparatus 186 acquires a list of segment addition data stored in the storage unit 192 via the communication unit 193 by the fourth specification accepting unit 196 of the analysis WS 184 and displays the acquired list of segment addition data on the display 506 or the like by the display unit 197. The user may view the displayed list of addition-average data and select addition-average data by using the keyboard 511 or the like. At this time, the biological-data processing apparatus 186 excludes the segment addition data that is recorded as an abnormal segment.

Subsequently, in step S111, the biological-data processing apparatus 186 performs an addition-averaging process on the selected at least one piece of segment addition data, according to the segment width. By this process, the biological-data processing apparatus 186 obtains addition-average data corresponding to at least one trigger.

In this manner, the biological-data processing apparatus 186 can obtain the addition-average data necessary for the process of estimating the action current by the analysis WS 4.

### <Example of Various Display Screens>

Next, various display screens displayed in the biological-data measurement system 181 will be described.

### (Example Screen for Specifying the Predetermined Count and the Total Addition Count)

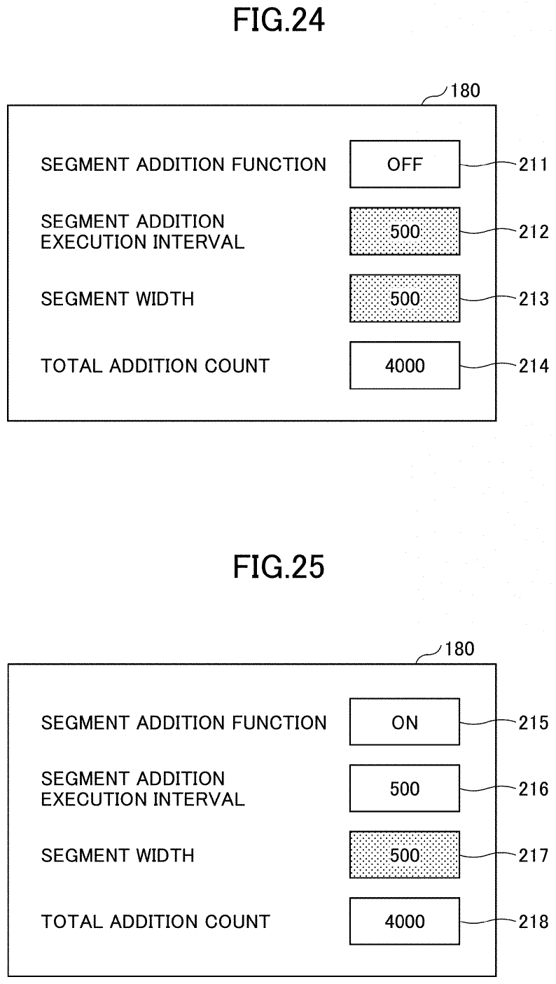

The predetermined count, the segment width, and the total addition count are set by using, for example, the screen of either FIG. 24 or FIG. 25 displayed on the display 506 or the like.

FIGS. 24 and 25 are diagrams illustrating a screen for specifying a predetermined count, a segment width, and a total addition count according to the present embodiment. In FIG. 24, a switch 211 is set as off so that segment addition is not performed. In this case, input boxes 212 and 213 are disabled so that the parameters associated with the segment addition-averaging process, such as the segment addition execution interval and the segment width, cannot be edited. The dot hatching in the input boxes 212 and 213 of FIG. 24 means that input 2C cannot be accepted. Hereinafter, boxes with dot hatching have the same meaning. An input box 214 is used for inputting the total addition count.

FIG. 25 is a diagram illustrating an example of a screen for specifying the interval of updating the predetermined count, a segment width, and a total addition count according to the present embodiment. In FIG. 25, a switch 215 is set as on so the addition count is automatically updated. Automatic updating is performed, and, therefore, in an input box 216, “500 times” is input as the automatic updating interval (update interval count) of the addition count. An input box 217 displays the same value as the automatic updating interval, and this value is specified as the segment width. An input box 218 is used for inputting the total addition count.

The counting WS 183 updates a predetermined count by adding “500 times” that is the addition execution interval count, to the predetermined count, every time the addition count reaches the predetermined count. In FIG. 25, 4000 times is specified as the total addition count, and, therefore, the predetermined count varies from 1000 times to 1500 times, . . . , 3500 times, and 4000 times by automatic updating, every time the addition count reaches the predetermined count.

The biological-data measurement system 181 performs a segment addition-averaging process every time the addition count reaches a predetermined count, and stores the segment addition data and information related to the segment addition in association with each other, in the data storage server 185.

### (Example of Measurement Screen and Operation Screen)

Next, the measurement screen and the operation screen will be described with reference to FIGS. 10 and 24 to 26.

FIG. 10 and the elements included in FIG. 10 are the same as those described in the first embodiment, and will not be described here.

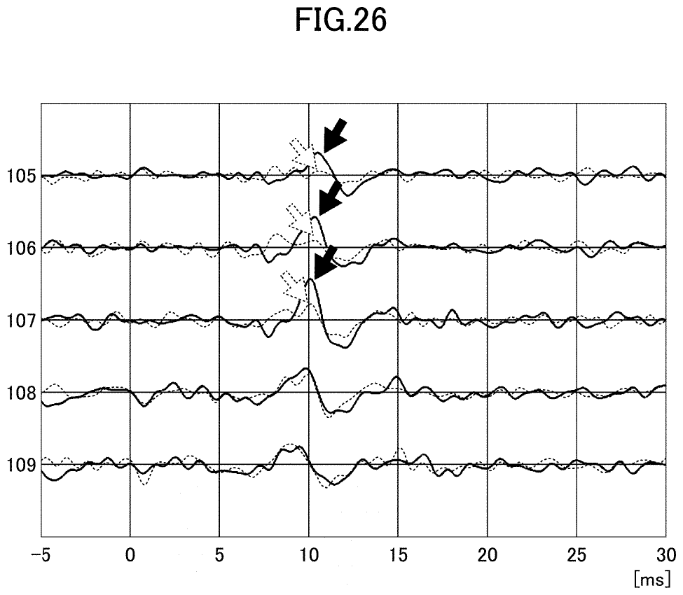

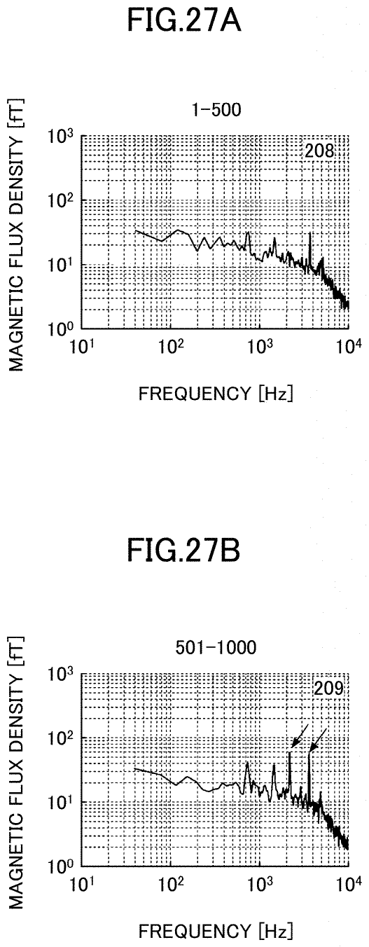

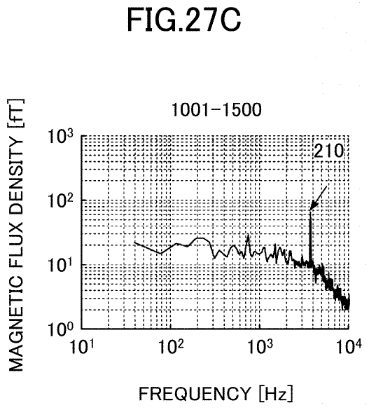

FIGS. 26 to 27C are diagrams illustrating an example of the display result of the segment addition data. FIG. 26 is a diagram for evaluating segment addition data as waveform data. FIGS. 27A to 27C are diagrams in which frequency analysis is applied to segment addition data, and a frequency spectrum is used for evaluation.

FIG. 26 illustrates an example in which the waveform data of a sensor that has particularly strongly detected a biological signal, is extracted from waveform data 63 in a case where two pieces of segment addition data are displayed, and the extracted pieces of waveform data are vertically arranged side-by-side and superimposed. The solid line graph illustrates segment addition data corresponding to the addition count of 1 to 500 times, and the dashed line graph illustrates segment addition data corresponding to the addition count of 501 to 1000 times. In the segment addition data corresponding to the addition count of 501 to 1000 times of the dashed line graph, the intensity of the biological signal has decreased at the point indicated by the arrow in the figure, and it is considered that some abnormality has occurred.

FIGS. 27A to 27C illustrate the display of results of frequency analysis performed on segment addition data corresponding to 1 to 500 times, segment addition data corresponding to 501 to 1000 times, and segment addition data corresponding to 1001 to 1500 times, respectively, as a frequency spectrum. There is no significant noise in the frequency spectrum of 1 to 500 times, but in the frequency spectrum of 501 to 1000 times and in the frequency spectrum of 1001 to 1500 times, peaks are present at a particular frequency, and it can be seen that some noise has entered the respective segments.

Accordingly, the user can determine that an abnormality has occurred in the segment of 501 to 1000 times and in the segment of 1001 to 1500 times. The analysis WS 184 may also superimpose and display the frequency spectrum corresponding to this segment addition data.

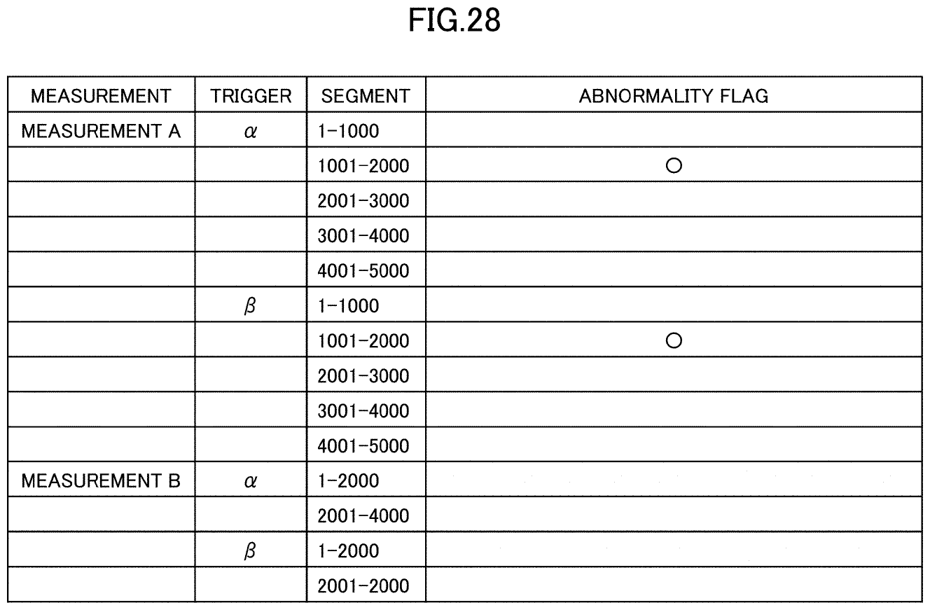

The display unit 197 displays an acquired list of addition-average data on the display 506. FIG. 28 illustrates an example of a list of addition-average data. Measurements A and B in FIG. 28 are information indicating the subjects that are living bodies. Trigger α and trigger β represent trigger signals each corresponding to stimulus applied to at least one part of the living body, and are examples of at least one trigger signal. The abnormality flag indicates that the user has specified that the corresponding segment addition data is abnormal.

When the user selects at least one piece of segment addition data from the displayed segment addition data list, the addition-averaging processing unit 194 performs the addition-averaging process according to the selected segment addition data based on the addition count corresponding to each selected segment, generates addition-average data, and stores the addition-average data in the data storage server 185. The estimating unit 195 can perform the estimation process by using the addition-average data generated by the procedure.

### <Effect of the Biological-Data Processing Apparatus 186>

As described above, the biological-data processing apparatus 186 performs a segment addition-averaging process by the addition-averaging processing unit 188 every time the addition count of biological data measured at a plurality of parts in the living body in response to a plurality of trigger signals, reaches a predetermined count, and the result of the process is displayed. The biological-data processing apparatus 186 stores the segment addition data, which is the result of the process, in the storage unit 192 in association with the addition count in the segment addition data.

Thereafter, the biological-data processing apparatus 186 performs addition-averaging by the addition-averaging processing unit 188 after excluding the segment that is determined to be abnormal by a user based on the displayed segment addition data, and obtains the addition-average data.

Thus, the biological-data processing apparatus 186 can display, evaluate, and add segment addition data to remove abnormal data and obtain addition-average data.

The biological-data processing apparatus 186 also includes the measurement control unit 189 for controlling whether the measurement by the measurement apparatus 182 is to be discontinued or extended, based on the display result of the segment addition data. Accordingly, when an abnormality has occurred in a segment that is a period during the measurement, the biological-data processing apparatus 186 can extend the measurement to supplement the measurement data of this segment or discontinue the measurement on the assumption that an abnormality has occurred in the measurement as a whole.

That is, the biological-data processing apparatus 186 can prompt the user to confirm, in the middle of the measurement, whether the measurement of a section in the middle of the measurement is performed normally or whether it is necessary to extend or discontinue the measurement, and can cause the measurement apparatus 182 to extend or discontinue the measurement according to the confirmation result. Thus, the biological-data processing apparatus 186 can ensure the initially planned addition count upon removing the data of the section where the abnormality has occurred.

### Fifth Embodiment

The biological-data measurement system 181 according to the fifth embodiment will be described. In the present embodiment, the predetermined count and the segment width for executing the segment addition-average data are independently specified, thereby improving the detection speed of detecting the abnormality. The overall configuration of the biological-data measurement system 181 is the same as that of the fourth embodiment, and, therefore, the descriptions will not be repeated here.

The biological-data measurement system 181 according to the fifth embodiment includes a measurement WS 183a, an analysis WS 184a, and a data storage server 185a. The measurement WS 183a, the analysis WS 184a, and the data storage server 185a configure a biological-data processing apparatus 186a.

The analysis WS 184a acquires all segment addition data by referring to the data storage server 185a and displays, by the display unit, the current distribution in the body estimated by the estimating unit based on the waveform data representing the segment addition data or the segment addition data. The analysis WS 184a detects incorrect segment addition data from on the displayed data, excludes detected segment addition data, and then performs addition-averaging on all segment addition data to obtain the addition-average data. The analysis WS 184a estimates the intensity of the action current based on the addition-average data obtained. The analysis WS 184a can display the estimation result on the display of the analysis WS 184a, transmit the estimation result to the data storage server 185 to be stored, or transmit the estimation result to an external device such as an external server.

The biological-data measurement system 181 may include apparatuses other than the measurement WS 183a, the analysis WS 184a, and the data storage server 185a in a communicable manner, or may include other biological-data measurement apparatuses other than the measurement apparatus 182 in a communicable manner.

<Example of Functional Configuration of the Biological-Data Processing Apparatus 186a>

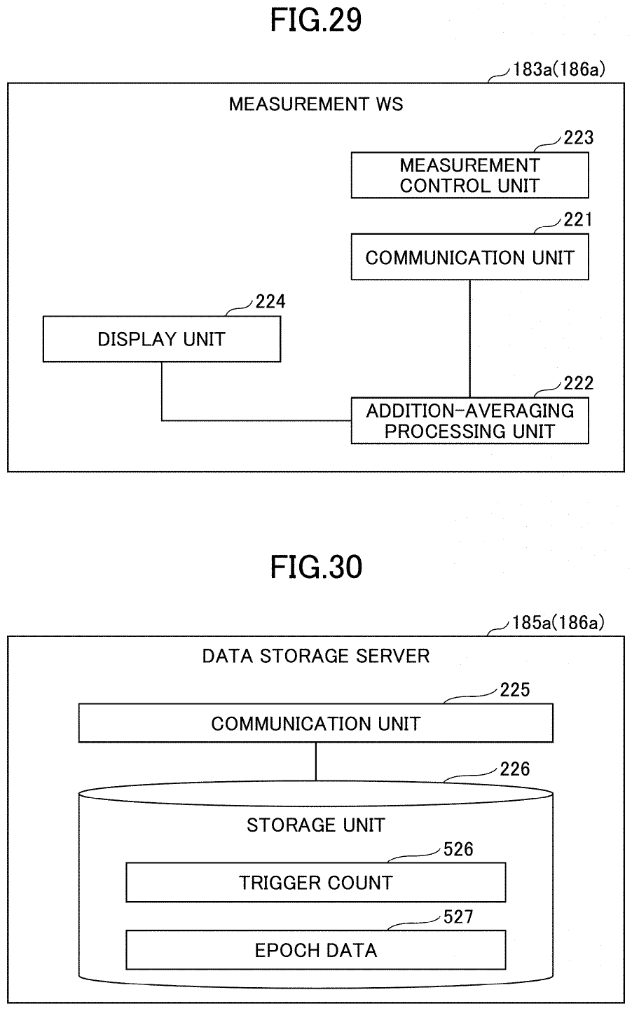

Referring to FIGS. 29 to 31, the functional configuration of the measurement WS 183a and the analysis WS 184a configuring the biological-data processing apparatus 186a will be described.

### (Example of Functional Configuration of the Measurement WS 183a)

FIG. 29 is a block diagram illustrating an example of a functional configuration of the measurement WS 183a. The measurement WS 183a includes a communication unit 221, an addition-averaging processing unit 222, a measurement control unit 223, and a display unit 224.

Each of these units is a function or functioning means implemented by one of the elements illustrated in FIG. 3 operating in response to an instruction from the CPU 501 according to a program loaded into the RAM 503 from the ROM 502. Although FIG. 29 illustrates the main configuration of the measurement WS 183a, the measurement WS 183a may have other configurations.

The communication unit 221 transmits and receives data and signals to and from the measurement apparatus 182, the analysis WS 184a, and the data storage server 185a.

The addition-averaging processing unit 222 acquires information on a predetermined count, a segment width, and a total addition count that are input by a user by using the keyboard 511 or the like (see FIG. 3). The total addition count is the total number of times that the addition-averaging processing unit 222 adds the biomagneticfield data. The addition-averaging processing unit 222 may acquire information on a predetermined count, a segment width, and a total addition count stored in advance in the HD 504 or the like by referring to the HD 504 or the like.

The addition-averaging processing unit 222 receives at least one trigger signal from the measurement apparatus 182 via the communication unit 221. Further, in response to at least one trigger signal, the addition-averaging processing unit 222 receives, via the communication unit 221, biomagneticfield data measured by the measurement apparatus 182 corresponding to a count that is obtained by counting back from the predetermined count by the segment width, and performs an addition-averaging process on the received biomagneticfield data.

The addition-averaging process is a process for calculating an average value by dividing, by the addition count, a value obtained by performing an addition process on the biomagneticfield data measured by the measurement apparatus 182 corresponding to a count that is obtained by counting back from the predetermined count by the segment width. The addition-averaging processing unit 222 associates the segment addition data with the addition count in the segment addition data and the addition data at the starting point and the ending point of the segment, and transmits the associated information to the data storage server 185a via the communication unit 221.

The measurement control unit 223 receives, via the communication unit 221, an instruction based on the evaluation result of evaluating the segment addition data by the analysis WS 184a, and can cause the measurement apparatus 182 to discontinue or extend the measurement based on the estimation result. The measurement control unit 223 can receive the instruction to discontinue or extend the measurement as the interrupt data at any time when the instruction is given.

### (Example of Functional Configuration of the Data Storage Server 185a)

FIG. 30 is a block diagram illustrating an example of a functional configuration of the data storage server 185a. As illustrated in FIG. 30, the data storage server 185a includes a communication unit 225 and a storage unit 226.

Each of these units is a function or functioning means implemented by one of the elements illustrated in FIG. 3 operating in response to an instruction from the CPU 501 according to a program loaded into the RAM 503 from the ROM 502. Although FIG. 30 illustrates the main configuration of the data storage server 185a, the data storage server 185a may have other configurations.

The communication unit 225 transmits and receives data and signals to and from the measurement WS 183a and the analysis WS 184a.

The storage unit 226 stores, in association with each other, a trigger count 526 and epoch data 527 received from the measurement WS 183a via the communication unit 225. The storage unit 226 also stores a list of segment addition data.

### (Example of Functional Configuration of the Analysis WS 184a)

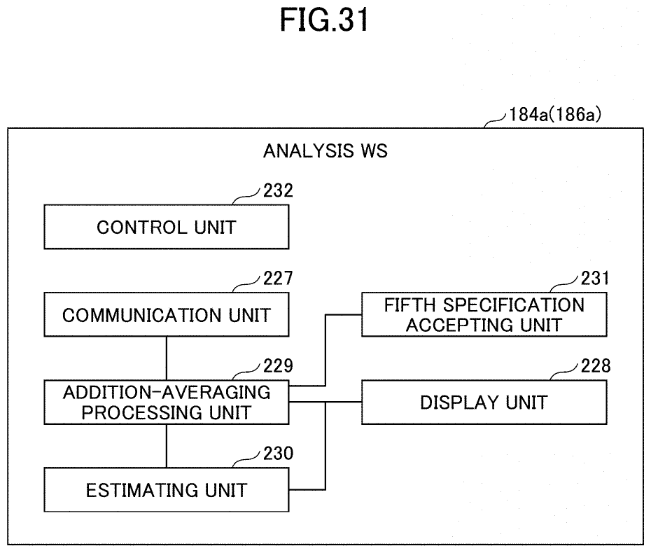

Next, FIG. 31 is a block diagram illustrating an example of a functional configuration of the analysis WS 184a. The analysis WS 184a includes a communication unit 227, an addition-averaging processing unit 229, an estimating unit 230, a display unit 228, a fifth specification accepting unit 231, and a measurement control unit 232.

Each of these units is a function or functioning means implemented by one of the elements illustrated in FIG. 3 operating in response to an instruction from the CPU 501 according to a program loaded into the RAM 503 from the ROM 502. Although FIG. 31 illustrates the main configuration of the analysis WS 184a, the analysis WS 184a may have other configurations.

The communication unit 227 transmits and receives data and signals to and from the measurement WS 183a and the data storage server 185a.

The addition-averaging processing unit 229 acquires a list of segment addition data stored in the storage unit 226 via the communication unit 227 and displays the acquired list of segment addition data on the display 506 by the display unit 228. The fifth specification accepting unit 231 can accept information on the addition count based on the result of the selection made by the user by viewing the list of the segment addition data. At least one piece of segment addition data selected is subjected to an addition-averaging process based on the addition count of each piece of data. The analysis WS 184a can exclude abnormal segment addition data from the list of segment addition data presented, and acquire addition-average data.

The estimating unit 230 is an example of a biological-data processing unit that performs processing based on biological data. The estimating unit 230 performs the process of estimating the intensity of the action current, based on the biomagneticfield data generated by the addition-averaging processing unit 229. The estimating unit 230 estimates the intensity of the action current by using the acquired addition-average data. As the estimation algorithm, it is possible to use “Array-Gain Constraint Minimum-Norm Spatial Filter With Recursively Updated Gram Matrix” or the like described above.

The display unit 228 displays the estimation result of the intensity of the action current estimated by the estimating unit 230. For example, the display unit 228 may display the estimation result on the display 506 to allow the user to view the estimation result. The display unit 228 can also display an analysis result such as waveform data, a frequency spectrum, or an estimation result representing the acquired addition-average data or segment addition data. According to the series of data acquisitions, the display process is performed even in the middle of the measurement.

When the analysis WS 184a displays the segment addition data as waveform data and a frequency spectrum on the display unit 228, the display unit 228 may display, in parallel, the segment addition data associated with a plurality of predetermined counts. The analysis WS 184a evaluates the displayed waveform data, determines whether the measurement has been performed normally in the segment, and records the determination result.

As to the determination of whether the measurement has been performed normally, the user may view the measurement result and input the determination result in the analysis WS 184a, or the analysis WS 184a may automatically make the determination according to some algorithm. For example, the user can view the frequency spectrum and determine that there is an abnormality when there is a peak at a position other than the a biological signal in the frequency, or determine that there is an abnormality when the amplitude of the biological signal obtained from the waveform data is obviously lowered in comparison to the amplitude of the other segment addition data. “Obviously lowered” means that the amplitude is lowered by an extent greater than or equal to the noise level included in the segment addition data, for example.

If it is determined that the segment addition data is abnormal data, the analysis WS 184a increases the addition count by an amount corresponding to the segment width, to exclude this abnormal segment addition data when generating the addition-average data. The analysis WS 184a accepts an instruction to extend the measurement given by a user by using the keyboard 511 or the like. The measurement control unit 232 may cause the measurement apparatus 182 to extend the measurement in response to this instruction. The analysis WS 184a may transmit the received instruction to extend the measurement, to the measurement WS 183a via the communication unit 227, and the measurement WS 183a may cause the measurement apparatus 182 to extend the measurement by the measurement control unit 223.

Even in the middle of the measurement, the estimating unit 230 estimates the intensity of the action current with respect to the segment addition data already stored, and the user can view the estimation result by the estimating unit 230 displayed by the display unit 228.

If the user determines that the amount of biomagneticfield data is insufficient, the user inputs an instruction to extend the measurement by using the keyboard 511 or the like. The measurement control unit 232 may cause the measurement apparatus 182 to extend the measurement in response to the instruction. Further, the analysis WS 184a may transmit the received instruction to extend the measurement, to the measurement WS 183a via the communication unit 227, and the measurement WS 183a may cause the measurement apparatus 182 to extend the measurement by the measurement control unit 223.

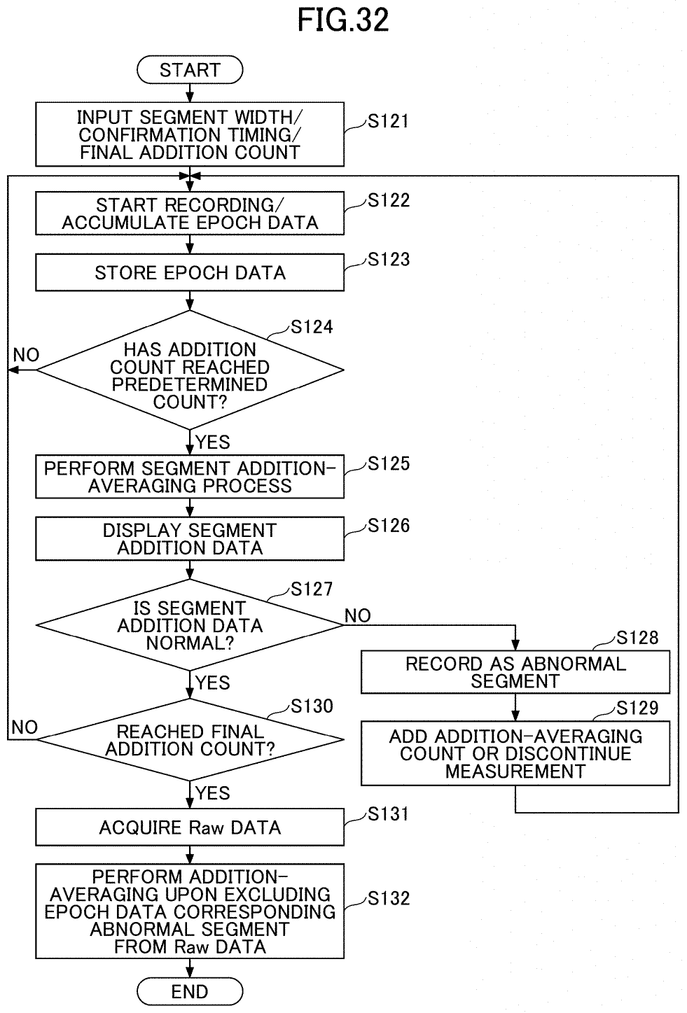

FIG. 32 is a flowchart illustrating an example of the operation of the biological-data processing apparatus 186a. Step S121 and step S122 in FIG. 32 are the same as step S101 and step S102 in FIG. 23. Step S124 and step S125 in FIG. 32 are the same as step S103 and step S104 in FIG. 23. Steps S127 through S130 in FIG. 32 are the same as steps S106 through S109 in FIG. 23. Hereinafter, the points that are different from FIG. 23 will be mainly described.

In step S123, the biological-data processing apparatus 186a transmits, by the measurement WS 183a, the epoch data to the data storage server 185a via the communication unit 221.

In step S126, the biological-data processing apparatus 186a transmits the segment addition data generated in step S125 to the display unit 224 to be displayed.

In step S131, the biological-data processing apparatus 186a acquires all epoch data (hereinafter, referred to as raw data) stored in the storage unit 226, by the fifth specification accepting unit 231 of the analysis WS 184a via the communication unit 227, and displays a list of epoch data included in the raw data on the display 506 or the like by the display unit 228. The user can view the displayed list of epoch data and select epoch data by using the keyboard 511 or the like. At this time, the analysis WS 184a excludes epoch data included in an abnormal segment.

In step S132, the analysis WS 184a performs an addition-averaging process on at least one piece of epoch data that is selected. By this process, the analysis WS 184a obtains addition-average data corresponding to at least one trigger.

In this manner, the analysis WS 184a can obtain the addition-average data necessary for the estimation process of estimating the action current.

### <Examples of Various Display Screens>

Various display screens displayed by the biological-data measurement system 181 will be described.

### (Example Screen for Specifying the Predetermined Count and the Total Addition Count)

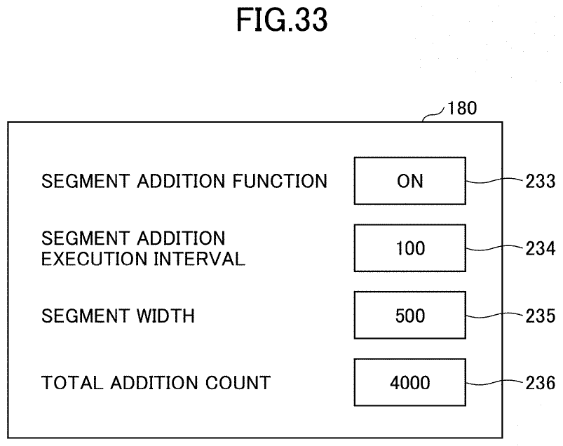

The predetermined count, the segment width, and the total addition count are set using, for example, the screen of FIG. 33 displayed on the display.

FIG. 33 is a diagram illustrating an example of a screen for specifying a predetermined count, a segment width, and a total addition count according to the present embodiment. In FIG. 33, a switch 233 is set as on to perform the automatic updating of the addition count. The automatic updating is to be performed, and, therefore, “100 times” is input in an input box 234 as the automatic updating interval (the update interval count) of the addition count, and “500 times” is input in an input box 235 as the segment width. An input box 236 is used for inputting the total addition count.

The measurement WS 183a updates the predetermined count by adding “100 times” that is the update interval count, to the predetermined count, every time the addition count reaches the predetermined count. In FIG. 33, 4000 times is specified as the total addition count, and, therefore, the predetermined count varies from 500 times to 600 times, 700 times, . . . , 3700 times, 3800 times, and 3900 times, by automatic updating, every time the addition count reaches the predetermined count.

The input box 235 is used for specifying the segment width. The segment width may be updated at any timing through the input box 235. At this time, the segment addition execution interval may also be changed as needed. Further, when the segment addition execution interval is smaller than the segment width, the same value as the segment width may be set as the initial predetermined count.

<Effect of the Biological-Data Processing Apparatus 186a>

As described above, the biological-data processing apparatus 186a performs a segment addition-averaging process by the addition-averaging processing unit 222 of the measurement WS 183a, every time the addition count of the biological data measured at a plurality of parts of a living body in response to a plurality of trigger signals, reaches a predetermined count, and displays the result of the segment addition-averaging process. The interval of the predetermined count is set independently from the segment width.

The biological-data processing apparatus 186a can set the interval of the predetermined count to be less than the segment width, and the latest data during the measurement can be confirmed earlier than in the fourth embodiment. Accordingly, when a certain abnormality occurs during the measurement, the biological-data processing apparatus 186a can detect an abnormality earlier, and can take measures such as discontinuing the measurement or extending the measurement upon taking countermeasures.

When an abnormality occurs, the biological-data processing apparatus 186a detects the abnormality earlier and ensures normal measurement upon taking countermeasures. Further, the epoch data in the section where the abnormality has occurred, can be precisely excluded.

### Sixth Embodiment

In the fourth and fifth embodiments, the evaluation of the segment addition data is performed at the measurement WS. The most important role of the measurement WS is to record the biomagneticfield data. The measurement WS is to avoid adverse effects such as omission in recording biomagneticfield data as a result of allocating the processing resources to display processing, etc., while confirming whether the measurement for each segment is performed normally.

A biological-data processing apparatus 186b according to the present embodiment integrates the function of displaying the segment addition data into an analysis WS 184b to achieve a function that is equivalent to that of the fourth embodiment while minimizing the processing performed at a measurement WS 183b. The functional configuration of the measurement WS 183b is the same as the functional configuration of the measurement WS 183a, the functional configuration of the analysis WS 184b is the same as the functional configuration of the analysis WS 184a, and the functional configuration of a data storage server 185b is the same as the functional configuration of the data storage server 185a.

<Example of Operation of the Biological-Data Processing Apparatus 186b>

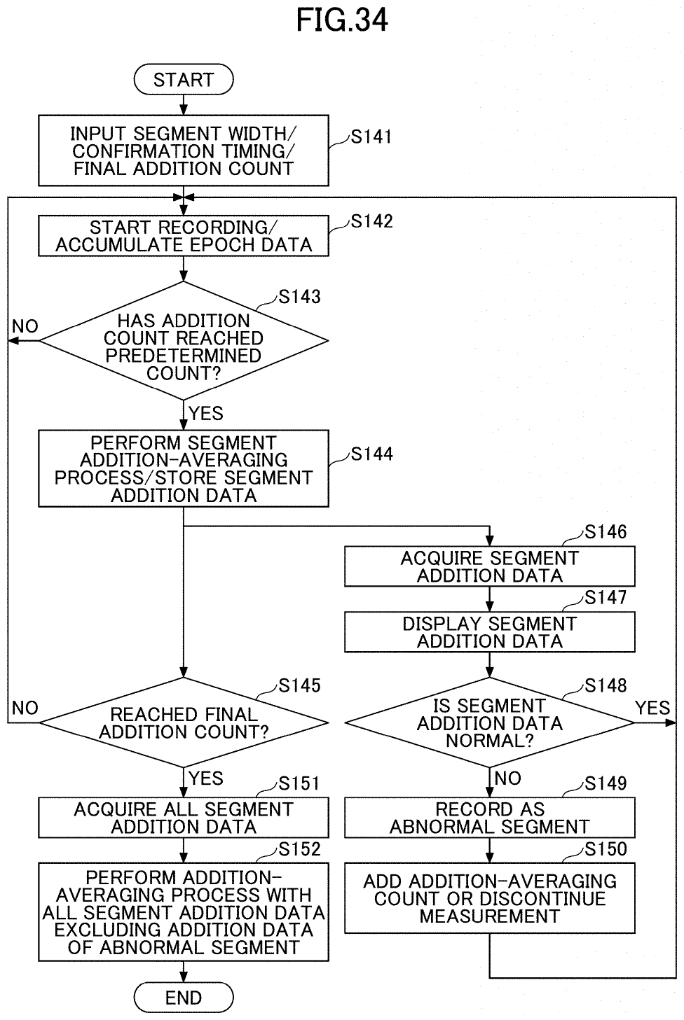

Referring now to FIG. 34, the operation of the biological-data processing apparatus 186b will be described.

First, in step S141, the biological-data processing apparatus 186b acquires, by the addition-averaging processing unit 222 of the measurement WS 183b, information on a predetermined count, a segment width, and a total addition count that the user has input by using the keyboard 511 or the like. The addition-averaging processing unit 222 may acquire information on the predetermined count, the segment width, and the total addition count stored in advance in the HD 504 or the like from the HD 504 or the like.

Subsequently, in step S142, the biological-data processing apparatus 186b receives and accumulates, by the addition-averaging processing unit 222, at least one trigger signal and biomagneticfield data measured in association with this trigger signal, from the measurement apparatus 182 via the communication unit 221.

Subsequently, in step S143, the biological-data processing apparatus 186b counts the received trigger signals, acquires the addition count for each trigger, and determines whether the addition count has reached a predetermined count, by the addition-averaging processing unit 222.

If it is determined that the addition count has reached a predetermined count (YES in step S143), in step S143, the biological-data processing apparatus 186b performs, by the addition-averaging processing unit 222, an addition-averaging process on the biomagneticfield data corresponding to a count that is obtained by counting back from the predetermined count by a the segment width. On the other hand, if it is determined that the addition count has not reached a predetermined count (NO in step S143), the biological-data processing apparatus 186b performs the operation of step S142 again.

Subsequently, in step S144, the biological-data processing apparatus 186b associates, by the addition-averaging processing unit 222, the segment addition data, which is the result of the segment addition-averaging process, with the information on the addition count in the segment addition data, and transmits the associated information to the data storage server 185b via the communication unit 221. The data storage server 185b stores the received segment addition data and the information related to the segment addition in association with each other. The segment addition data is transmitted to the display unit 224 to be displayed.

Subsequently, in step S145, the biological-data processing apparatus 186b determines whether the addition count has reached the final addition count (the total addition count) by the measurement control unit 223. The determination may also be made by the addition-averaging processing unit 222 instead of the measurement control unit 223.

If it is determined that the addition count has not reached the final addition count (NO in step S145), the biological-data processing apparatus 186b performs the operation from step S142 again. On the other hand, if it is determined that the addition count has reached the final addition count (YES in step S145), the biological-data processing apparatus 186b causes the measurement apparatus 182 to end the measurement, by the measurement control unit 223.

In this manner, the biological-data processing apparatus 186b can perform the addition-averaging process by the measurement WS 183b and control the measurement apparatus 182 in response to an instruction to discontinue or extend the measurement.

Subsequently, in step S146, the biological-data processing apparatus 186b acquires a list of segment addition data stored in the storage unit 226 via the communication unit 227 by the fifth specification accepting unit 231 of the analysis WS 184b.

Subsequently, in step S147, the biological-data processing apparatus 186b displays the acquired list of segment addition data on the display 506 or the like by the display unit 228. The user views the displayed list of segment addition data, specifies any segment addition data, and causes the display unit 228 to display waveform data, a frequency spectrum, an estimation result, or the like, of the biomagneticfield data obtained as a result of applying some kind of an analysis process.

Subsequently, in step S148, the biological-data processing apparatus 186b confirms the segment addition data displayed on the display unit 228 and accepts an instruction from a user who has determined whether the segment addition data is normal.

In step S148, if it is determined that the segment addition data is not normal (NO in step S148), in step S149, the biological-data processing apparatus 186b records the segment as an abnormal segment, and in step S150, the biological-data processing apparatus 186b gives an instruction to increase the final addition count, i.e., to extend the measurement, or to discontinue the measurement. The instruction is transmitted to the measurement WS 183b via the communication unit 227, and a process corresponding to the instruction is performed immediately.

The biological-data processing apparatus 186b repeats the processing of step S146 through step S150 until the final addition count is reached.

In contrast, after it is determined that the final addition count is reached in step S145, in step S151, the biological-data processing apparatus 186b acquires a list of segment addition data stored in the storage unit 226 via the communication unit 227 by the fifth specification accepting unit 231 and displays the acquired list of the segment addition data on the display 506 or the like by the display unit 228. At this time, the biological-data processing apparatus 186b excludes segment addition data recorded as an abnormal segment.

Subsequently, in step S152, the biological-data processing apparatus 186b performs an addition-averaging process on the selected at least one piece of segment addition data according to the segment width. By this process, addition-average data corresponding to at least one trigger is obtained.

In this manner, the biological-data processing apparatus 186b can obtain the addition-average data necessary for performing the process of estimating the action current by the analysis WS 184b.

<Effect of the Biological-Data Processing Apparatus 186b>

As described above, the biological-data processing apparatus 186b performs a segment addition-averaging process by the addition-averaging processing unit 222 every time the addition count of the biological data, which is measured in response to a plurality of trigger signals, reaches a predetermined count. The biological-data processing apparatus 186b stores the segment addition data, which is the result of the processing, in the storage unit 226 in association with the addition count in the segment addition data.

Thereafter, the segment data is evaluated in the analysis WS 184b to determine whether the measurement in the corresponding segment has been normally performed. After the measurement ends, the biological-data processing apparatus 186b performs addition-averaging upon excluding the segment that is determined to be abnormal by the user, to obtain the addition-average data.

Thus, the biological-data processing apparatus 186b can display, evaluate, and add segment addition data in the analysis WS 184b to remove abnormal data and obtain addition-average data. Thus, the biological-data processing apparatus 186b can assure signal quality by removing data of the section where an abnormality has occurred while minimizing the processing load on the measurement WS 183b.

The embodiments illustrated above do not exclude each other.

The biological-data processing apparatus, the biological-data measurement system, and the recording medium are not limited to the specific embodiments described in the detailed description, and variations and modifications may be made without departing from the scope of the present invention.

Embodiments also include a program. For example, a program, stored in a non-transitory computer-readable recording medium, causes a computer to execute a process. The process includes performing an addition-averaging process every time an addition count of biological data reaches a predetermined count, the biological data being measured in response to a trigger signal associated with a stimulus applied to one or more parts; storing, in a storage, addition-average data resulting from the addition-averaging process performed for each of the stimulated one or more parts, or segment addition data, in association with the addition count in the addition-average data or the segment addition data; and performing a biological data process based on the biological data, by using the addition-average data corresponding to each of the stimulated one or more parts, the addition-average data being acquired by referring to the storage based on the addition count that is specified. By such a program, the same effect as the biological-data processing apparatus described above can be attained.

The functions of each of the embodiments described above may be implemented by one or more processing circuits. As used herein, a “processing circuit” includes a processor programmed to execute each function by software such as a processor implemented in an electronic circuit; or devices such as an Application Specific Integrated Circuit (ASIC) a digital signal processor (DSP), a field programmable gate array (FPGA), and a conventional circuit module, designed to execute each function as described above.

According to one embodiment of the present invention, the effect of the addition-averaging process can be equally attained for each of the stimulated parts corresponding to a trigger signal, when the addition-averaging process is performed on a plurality of pieces of biological data measured in response to a trigger signal associated with a stimulus applied to a plurality of parts.

What is claimed is:

1. A biological-data processing apparatus comprising:
a processor; and
a memory that includes instructions, which when executed, cause the processor to execute:
performing an addition-averaging process every time an addition count of biological data reaches a predetermined count, the biological data being measured in response to a trigger signal associated with a stimulus applied to one or more parts;
storing, in a storage, addition-average data resulting from the addition-averaging process performed for each of the stimulated one or more parts, in association with the addition count in the addition-average data; and
performing a biological data process based on the biological data, by using the addition-average data corresponding to each of the stimulated one or more parts, the addition-average data being acquired by referring to the storage based on the addition count that is specified.

2. The biological-data processing apparatus according to claim 1, wherein
the biological data is biomagneticfield data of a biomagneticfield generated by a living body, and
the biological data process is performed by estimating an intensity of an action current for each of the stimulated one or more parts in the living body, based on the biomagneticfield data.

3. The biological-data processing apparatus according to claim 1, wherein the processor is further caused to execute:
accepting a specification of the addition count.

4. The biological-data processing apparatus according to claim 3, wherein the processor is further caused to execute:
accepting a specification of the predetermined count.

5. The biological-data processing apparatus according to claim 3, wherein the processor is further caused to execute:
accepting a specification of an update interval count, wherein
the predetermined count is automatically updated to a count obtained by adding the accepted update interval count to the predetermined count, every time the addition count reaches the predetermined count.

6. The biological-data processing apparatus according to claim 1, wherein the processor is further caused to execute:
switching a method of performing the biological data process according to the addition count.

7. The biological-data processing apparatus according to claim 6, wherein
the switching includes switching the method only in response to determining that the addition count corresponds to a total addition count,
the biological data process is performed at a high speed compared to a case where the addition count corresponds to the total addition count, in response to determining that the addition count does not correspond to the total addition count, and
the biological data process is performed with high precision compared to a case where the addition count does not correspond to the total addition count, in response to determining that the addition count corresponds to the total addition count.

8. A biological-data measurement system comprising:
the biological-data processing apparatus according to claim 1; and
a measurement apparatus configured to perform measurement of a living body.

9. The biological-data measurement system according to claim 8, wherein the processor is further caused to execute:
controlling the measurement apparatus to discontinue or extend the measurement based on a processing result of the biological data process.

10. The biological-data measurement system according to claim 9, wherein the processor is further caused to execute:
displaying, on a display, the processing result of the biological data process,
accepting an instruction to either discontinue or extend the measurement by the measurement apparatus, and
controlling the measurement apparatus to discontinue or extend the measurement according to the instruction.

11. A non-transitory computer-readable recording medium storing a program that causes a computer to execute a process, the process comprising:
performing an addition-averaging process every time an addition count of biological data reaches a predetermined count, the biological data being measured in response to a trigger signal associated with a stimulus applied to one or more parts;
storing, in a storage, addition-average data resulting from the addition-averaging process performed for each of the stimulated one or more parts, in association with the addition count in the addition-average data; and
performing a biological data process based on the biological data, by using the addition-average data corresponding to each of the stimulated one or more parts, the addition-average data being acquired by referring to the storage based on the addition count that is specified.

12. A biological-data processing apparatus comprising:
a processor; and
a memory that includes instructions, which when executed, cause the processor to execute:
performing a segment addition-averaging process on biological data every time an addition count of the biological data according to a trigger signal reaches a predetermined count;
storing, in a storage, segment addition data resulting from a plurality of the segment addition-averaging processes, in association with the addition count in the segment addition data;
displaying, on a display, the segment addition data; and
performing an addition-averaging process to acquire addition-average data, the addition-averaging process being performed on any piece of the segment addition data among the segment addition data acquired by referring to the storage.

13. The biological-data processing apparatus according to claim 12, wherein the processor is further caused to execute:
performing a biological data process based on the biological data, with respect to at least one of the segment addition data or the addition-average data.

14. The biological-data processing apparatus according to claim 13, wherein
the biological data is biomagneticfield data of a biomagneticfield generated by a living body, and
the biological data process includes at least one of a process of estimating an intensity of an action current in the living body based on the biomagneticfield data or a process of performing frequency analysis on the biomagneticfield data, and
the displaying includes displaying a result of the biological data process on the display.

15. The biological-data processing apparatus according to claim 14, wherein
the displaying includes displaying a plurality of pieces of the segment addition data side-by-side with a plurality of pieces of the addition-average data.

16. A biological-data processing apparatus comprising:
a processor; and
a memory that includes instructions, which when executed, cause the processor to execute:
performing a segment addition-averaging process on biological data every time an addition count of the biological data according to a trigger signal reaches a predetermined count;
displaying, on a display, segment addition data resulting from the segment addition-averaging process;
storing, in a storage, all epoch data associated with the trigger signal, and
performing an addition-averaging process to acquire addition-average data, the addition-averaging process being performed upon selecting any piece of epoch data among all of the epoch data acquired by referring to the storage.

17. The biological-data processing apparatus according to claim 16, wherein the predetermined count and a segment width are set separately.
